(19) United States
(12) Patent Application Publication
(10) Pub. No.: US 2018/0057457 A1
(43) Pub. Date: Mar. 1, 2018

(54) Chemical Process for Preparing Pyrimidine Derivatives and Intermediates Thereof

(71) Applicant: Novartis AG, Basel (CH)

(72) Inventors: Mark Clinton Davis, Basel (CH); Baoqing Gong, Basel (CH); Denis Har, East Hanover, NJ (US); Tobias Kapferer, Basel (CH); Xuchun Zheng, Hangzhou (CN)

(73) Assignee: Novartis AG, Basel (CH)

(21) Appl. No.: 15/555,103

(22) PCT Filed: Mar. 4, 2015
(86) PCT No.: PCT/CN2015/073647

§ 371 Date: Sep. 1, 2017

(51) Int. Cl. C07D 211/28; C07D 211/82; C07D 239/42

(52) U.S. Cl. CPC C07D 211/28; C07D 239/42; C07D 211/82

(57) ABSTRACT

The present disclosure relates to a method of synthesizing 5-chloro-N2-(2-isopropoxy-5-methyl-4-(piperidin-4-yl)phenyl)-N4-[2-(propane-2-sulfonyl)-phenyl]-pyrimidine-2,4-diamine (ceritinib) and/or intermediates thereof, their use as pharmaceuticals and pharmaceutical compositions and the use of intermediates for preparing ceritinib.

### FIELD OF THE DISCLOSURE

The present disclosure is in the field of organic synthesis and is directed to a method of synthesizing 5-chloro-N2-(2-isopropoxy-5-methyl-4-(piperidin-4-yl)phenyl)-N4-[2-(propane-2-sulfonyl)-phenyl]-pyrimidine-2,4-diamine (ceritinib) and/or intermediates thereof, methods for further preparing pharmaceuticals and pharmaceutical compositions from ceritinib or from intermediates, the use of intermediates for preparing ceritinib and intermediates themselves.

### BACKGROUND OF THE DISCLOSURE

Anaplastic Lymphoma Kinase (ALK) is a member of the insulin receptor superfamily of receptor tyrosine kinases. Chromosomal rearrangements involving ALK has been detected in a variety of human malignancies, such as oncogenesis in hematopoietic and non-hematopoietic tumors, leading to disturbances in the regulation pathway of the cells. Inhibition or suppression of the ALK pathways using an ALK tyrosine kinase inhibitor engenders the cell growth arrest and apoptosis of malignant cells. The study of ALK fusion proteins has also raised the possibility of new therapeutic treatments for patients with ALK positive malignancies.

Ceritinib is an ALK inhibitor with the chemical formula 5-chloro-N2-(2-isopropoxy-5-methyl-4-(piperidin-4-yl)phenyl)-N4-[2-(propane-2-sulfonyl)-phenyl]-pyrimidine-2,4-diamine. A method for preparing it was disclosed in WO2008/073687.

### SUMMARY OF THE DISCLOSURE

Chemical processes are usually carried out on a small scale in a research/early development phase, and the scale successively increases in late phase development to finally reach the full size production scale. Upon scaling up a process, topics related to process safety are becoming more and more important. Failure to scale up properly may lead to the loss of process control and accidents, such as unexpected exothermic reactions (runaway reactions), health hazards while handling large amount of hazardous and/or toxic chemicals or environmental hazards.

Surprisingly it was found that the process to synthesize ceritinib (5-chloro-N2-(2-isopropoxy-5-methyl-4-(piperidin-4-yl)phenyl)-N4-[2-(propane-2-sulfonyl)-phenyl]-pyrimidine-2,4-diamine) and the intermediates thereof can be prepared with a cost efficient and safer method. Therefore the present disclosure is directed to a new synthesis of ceritinib and its intermediates, using less hazardous chemicals and/or reaction conditions, generating less waste and providing a reproducible process that is easier to handle on a larger scale, a process that is more efficient and generates better quality compounds.

The first aspect of the present disclosure is a compound of formula (C2-1)

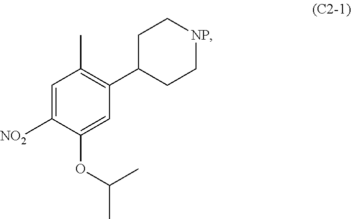

wherein P is a protecting group.

A further aspect of the disclosure provides a process for preparing a compound of formula (C2-1), comprising reacting a compound of formula (A)

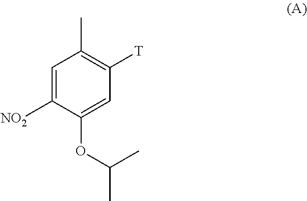

with a compound of formula (B) in a solvent

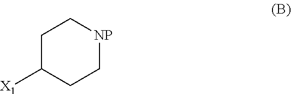

in the presence of at least one catalyst, and optionally a co-catalyst or additive, wherein
P is a protecting group; and
T and X1 can be independently selected from the group consisting of Cl, Br, I, OTf, OTs, OPiv, MgCl, MgBr, MgI, Sn(Alkyl)₃, Si(Alkyl)₃, Si(OAlkyl)₃, ZnCl, ZnBr, ZnI, Zn(Alkyl), B(OH)₂, B(OC(CH₃)₂C(CH₃)₂O), 9-BBN, B(Sia)₂, B(Cat), B(Cy)₂, BF₃⁻, B(MIDA).

A further aspect of the disclosure relates to a process for preparing a compound of formula (C2-1), comprising the steps of reacting a compound of formula (C2-3)

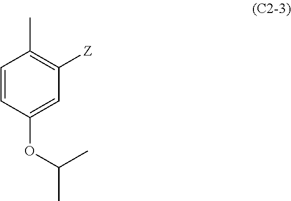

with

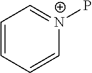

(pyridinium-P) in a solvent to produce a compound (C2-2);

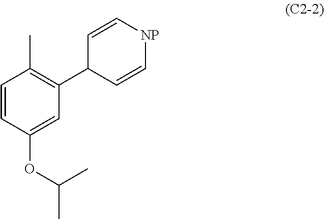

and transforming the compound (C2-2) to form the compound of formula (C2-1)

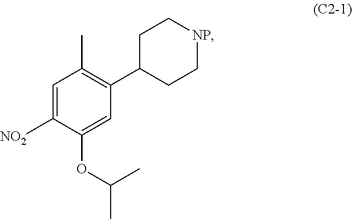

wherein
P is a protecting group and
Z is selected from MgCl, MgBr, MgI, ZnCl, ZnBr, ZnI, Zn(Alkyl). The compound (C2-2) is transformed by reduction and nitration to yield the compound of formula (C2-1).

Another aspect of this disclosure relates to a process for preparing a compound of formula (C2), or a salt thereof,

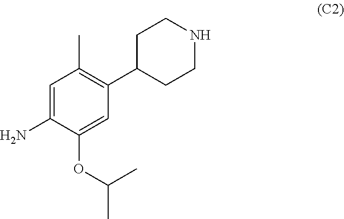 [SMILES: Cc1cc(N)c(OC(C)C)cc1C1CCNCC1]

the process comprising reduction and deprotection of the compound of formula (C2-1).

Yet another aspect of this disclosure relates to the process for preparing a compound of formula (C2), or a salt thereof, the process comprising a step of reducing a compound of formula (C) in a solvent

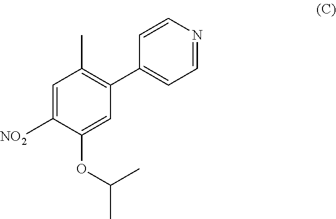 [SMILES: Cc1cc([N+](=O)[O-])c(OC(C)C)cc1-c1ccncc1]

in the presence of at least one catalyst and hydrogen.

Further aspect of the present disclosure relates to a one-pot process for preparing a compound of formula (C), or a salt thereof, the process comprising the steps of reacting a compound of formula (AA) in a solvent

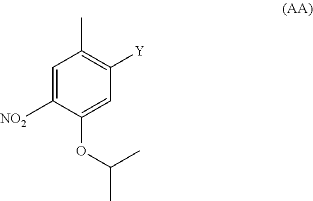

with a compound of formula D in the presence of X₃B(X₂)₂, at least one catalyst, a base and optionally a ligand,

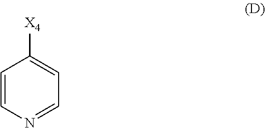

without isolating a compound of formula (E), wherein

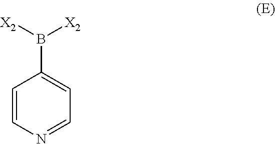

Y is selected from Br, Cl, I, OTf, OTs, OPiv and OMs;
X₄ is selected from Br, Cl, I, OTf, OTs, OPiv and OMs;
B(X₂)₂ is selected from B(OH)₂, B(OC(CH₃)₂C(CH₃)₂O), 9-BBN, B(Sia)₂, B(cat), B(Cy)₂, BF₃⁻ and B(MIDA); and
X₃ is H, B(X₂)₂.

A further aspect of the disclosure provides a compound of formula (C3-1)

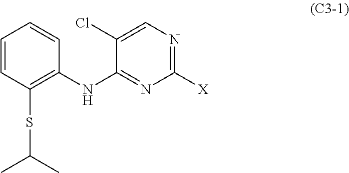

wherein X is selected from halogen (F, Cl, Br, I), alkoxy, aryloxy, alkylthio, sulfinyl and sulfonyl.

Another aspect of the disclosure provides the process for preparing a compound of formula (C3-1), the process comprising the step of reacting a compound of formula (F)

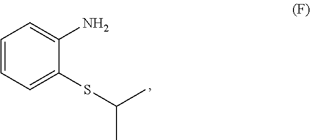

or a salt thereof,
with a compound of formula (G)

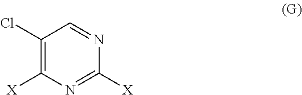

in the presence of a base and optionally in a solvent, wherein X is selected from halogen (F, Cl, Br, I), alkoxy, aryloxy, alkylthio, arylthio, sulfinyl, sulfonyl.

Another aspect of the disclosure relates to a process for preparing a compound of formula (C3), comprising the process for oxidizing (C3-1)

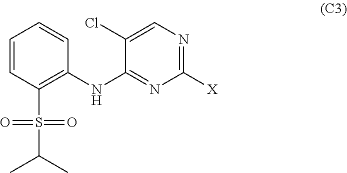

in a solvent, wherein X is selected from halogen (F, Cl, Br, I), alkoxy, aryloxy, alkylthio, sulfinyl, sulfonyl.

A further aspect of the disclosure provides a process for preparing a compound of formula (C3), the process comprising the steps of (i) reacting a compound of formula (H)

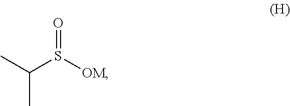

with a compound of formula (I), wherein M is selected from Li, Na, K, 0.5 Zn, 0.5 Ca, to obtain an intermediate;

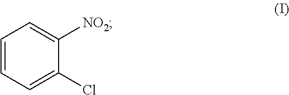

(ii) reducing the intermediate; and
(iii) reacting the reduced intermediate with a compound of formula (G)

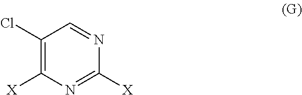

in the presence of a base, wherein X is selected from the group consisting of halogen (F, Cl, Br I), alkoxy, aryloxy, alkylthio, sulfinyl, sulfonyl.

Another aspect of the disclosure relates to a process for preparing ceritinib, or a salt thereof, the process comprising the steps of:
i. preparing a compound of formula (C2-1)
ii. preparing a compound of formula (C2), or a salt thereof,
iii. providing a compound (C3), and
iv. reacting a compound of formula (C2), or a salt thereof, with a compound of formula (C3), to obtain ceritinib, or a salt thereof.

A further aspect of the disclosure relates to a process for preparing ceritinib, or a salt thereof, the process comprising the steps of:
(a) preparing a compound of formula (C2), or a salt thereof,
(b) providing a compound of formula (C3), and
(c) reacting the compound of formula (C2), or a salt thereof, with a compound of formula (C3), to obtain ceritinib, or a salt thereof.

Another aspect of the disclosure provides a process for preparing ceritinib, or a salt thereof, the process comprising the steps of:
(aa) preparing a compound of formula (C2), or a salt thereof,
(bb) providing a compound of formula (C3), and
(cc) reacting the compound of formula (C2), or a salt thereof, with a compound of formula (C3), to obtain ceritinib, or a salt thereof.

Another aspect of the disclosure provides a process for preparing ceritinib, or a salt thereof, comprising
(I) providing a compound of formula (C2), or a salt thereof,
(II) preparing a compound of formula (C3-1)
(III) preparing from the compound of formula (C3-1) the compound of formula (C3), and
(IV) reacting the compound of formula (C2), or a salt thereof, with the compound of formula (C3), to obtain ceritinib, or a salt thereof.

A further aspect of the disclosure provides the use of a compound of formula (C2-1) for preparing ceritinib, or a salt thereof.

A further aspect of the disclosure provides the use of a compound of formula (C3-1) for preparing ceritinib, or a salt thereof.

Another aspect of the disclosure is a compound of formula (C2-2)

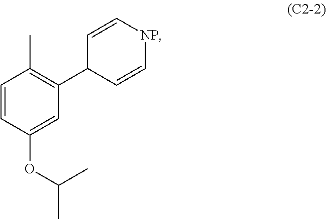

wherein
P is a protecting group.

A further aspect of the disclosure is the use of compound of formula (C2-2) for preparing ceritinib, or a salt thereof.

### DETAILED DESCRIPTION OF THE DISCLOSURE

It was observed that increasing the amount of reactants and solvent in order to scale up a reaction to a full size production plant may engender some risks such as loss of process control, unexpected exothermic reactions, accidents and safety issues while handling large amount of hazardous and/or toxic chemicals.

Surprisingly, it was found that modifying the process to synthesize ceritinib (5-chloro-N2-(2-isopropoxy-5-methyl-4-(piperidin-4-yl)phenyl)-N4-[2-(propane-2-sulfonyl)-phenyl]-pyrimidine-2,4-diamine) and intermediates provides a scalable method that can safely be handled on a larger scale with reproducible yields, less hazardous/toxic chemicals and produces less waste. In addition, this process produces more efficiently better quality compounds, at a lower cost. A summary of the process is showed in Scheme 1, vide infra.

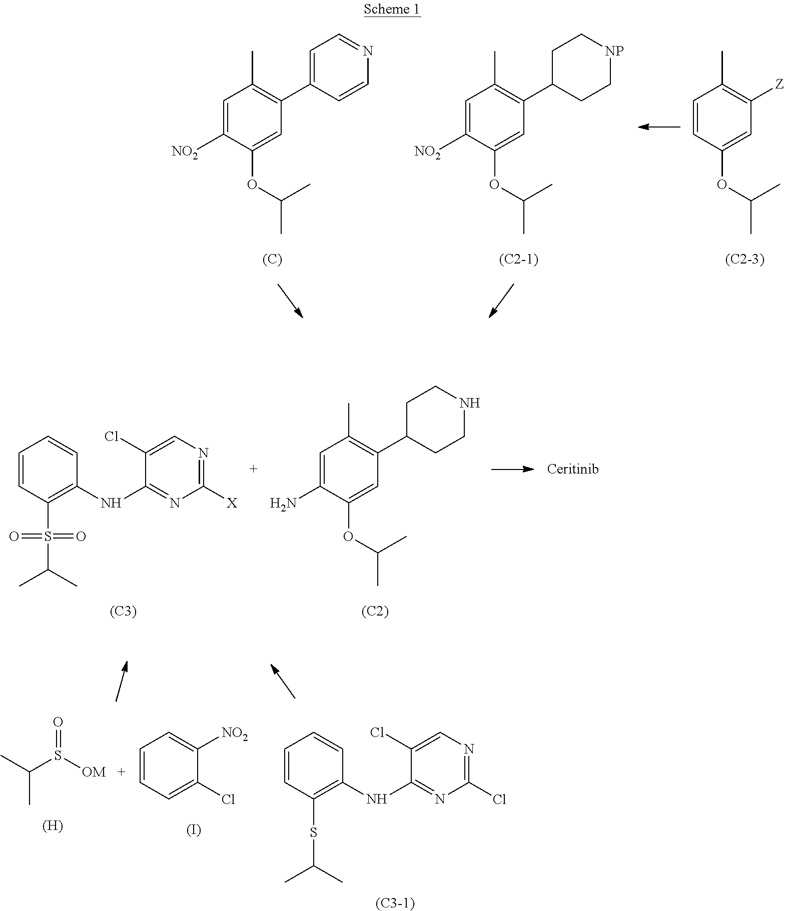

### 1.1 Compound of Formula (C2-1):

The first aspect of the present disclosure relates to an intermediate compound—a compound of formula (C2-1)

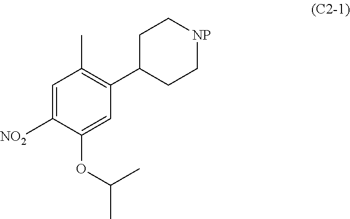

wherein P is a protecting group, or in this case a nitrogen protecting group. The compound of formula (C2-1) can be used for preparing ceritinib.

The term “protecting group” or “nitrogen protecting group” may be present and should protect the functional groups concerned against unwanted secondary reactions, such as acylations, etherifications, esterifications, oxidations, solvolysis and similar reactions. It is a characteristic of protecting groups that they lend themselves readily, i.e. without or with very limited undesired secondary reactions, to removal, typically by solvolysis, reduction, photolysis or also by enzyme activity, for example under conditions analogous to physiological conditions, and that they are not present in the end-products. The specialist knows, or can easily establish, which protecting groups are suitable with the reactions mentioned hereinabove and hereinafter. Preferably, if two or more protecting groups are present in one intermediate mentioned, they are chosen so that, if one of the groups needs to be removed, this can be done selectively, e.g. using two or more different protecting groups that are cleavable under different conditions, e.g. one class by mild hydrolysis, the other by hydrolysis under harder conditions, one class by hydrolysis in the presence of an acid, the other by hydrolysis in the presence of a base, or one class by reductive cleavage (e.g. by catalytic hydrogenation), the other by hydrolysis, or the like. Suitable nitrogen protecting groups are conventionally used in peptide chemistry and are described e.g. in the relevant chapters of standard reference works such as J. F. W. McOmie, “Protective Groups in Organic Chemistry”, Plenum Press, London and New York 1973; T. W. Greene and P. G. M. Wuts, “Greene's Protective Groups in Organic Synthesis”, Fourth Edition, Wiley, New York 2007; in “The Peptides”; Volume 3 (editors: E. Gross and J. Meienhofer), Academic Press, London and New York 1981, and in “Methoden der organischen Chemie” (Methods of Organic Chemistry), Houben Weyl, 4th edition, Volume 15/l, Georg Thieme Verlag, Stuttgart 1974. Preferred nitrogen protecting groups generally comprise: C₁-C₆-alkyl, preferably C₁-C₄-alkyl, more preferably C₁-C₂-alkyl, (e.g. acetyl, allyl, tertbutyl) most preferably C₁-alkyl which is mono-, di- or tri-substituted by trialkylsilyl-C₁-C₇-alkoxy (eg. trimethylsilyethoxy), aryl, preferably phenyl, or an heterocyclic group (e.g., benzyl, cumyl, benzhydryl, pyrrolidinyl, trityl, pyrrolidinylmethyl, 1-methyl-1,1-dimethylbenzyl, (phenyl)methylbenzene) wherein the aryl ring or the heterocyclic group is unsubstituted or substituted by one or more, e.g. two or three, residues, e.g. selected from the group consisting of C₁-C₇-alkyl, hydroxy, C₁-C₇-alkoxy, C₂-C₈-alkanoyl-oxy, halogen, nitro, cyano, and CF₃; aryl-C₁-C₂-alkoxycarbonyl (preferably phenyl-C₁-C₂-alkoxycarbonyl (eg. benzyloxycarbonyl (Cbz), benzyloxymethyl (BOM), pivaloyloxymethyl (POM)); C₁-C₁₀-alkenyloxycarbonyl; C₁-C₆alkylcarbonyl (eg. acetyl or pivaloyl); C₆-C₁₀-arylcarbonyl; C₁-C₆-alkoxycarbonyl (eg. tertbutoxycarbonyl (Boc), methylcarbonyl, trichloroethoxycarbonyl (Troc), pivaloyl (Piv), allyloxycarbonyl); C₆-C₁₀-arylC₁-C₆-alkoxycarbonyl (e.g. 9-fluorenylmethyloxycarbonyl (Fmoc)); allyl or cinnamyl; sulfonyl or sulfenyl; succinimidyl group, silyl groups (e.g. triarylsilyl, trialkylsilyl, triethylsilyl (TES), trimethylsilylethoxymethyl (SEM), trimethylsilyl (TMS), triisopropylsilyl or tertbutyldimethylsilyl). According to the disclosure the preferred protecting group (P) can be selected from the group consisting of tert-butyloxycarbonyl (Boc), benzyloxycarbonyl, methyloxycarbonyl or benzyl.

A compound of formula (C2-1) can be prepared by a process which comprises reacting a compound of formula (A) with a compound of formula (B) in a solvent in the presence of at least one catalyst, optionally a co-catalyst or additive, as defined in Scheme 2. The reaction is normally heated. Suitable solvents used for the reaction are, for example, tetrahydrofuran (THF), 2-methyl-tetrahydrofuran, 1,4-dioxane, diethyl ether, toluene, dimethylformamide (DMF), dimethylacetamide (DMA), dimethylsulfoxide (DMSO), dimethoxyethane (DME), dichloromethane, N-methyl-2-pyrrolidone (NMP), 1-butyl-2-pyrrolidone (NBP), acetonitrile, acetone, ethylacetate, isopropylacetate, tertbutylacetate, pentane, hexane, heptane, anisole, pyridine, triethylamine, dimethycarbonate, water, methanol, ethanol, n-propanol, 2-propanol, n-butanol, 2-butanol, tert-butanol, or mixtures thereof.

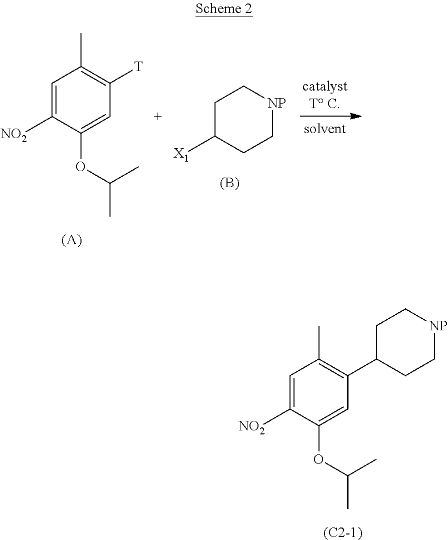

Although selection of a suitable protecting group (P) is vast, the preferred protecting group (P) is tert-butyloxycarbonyl (Boc), benzyloxycarbonyl, methyloxycarbonyl, ethyloxycarbonyl, allyloxycarbonyl, phenyloxycarbonyl, formyl, acetyl or benzyl. The most preferred protecting group in this process is is tert-butyloxycarbonyl (Boc), benzyloxycarbonyl, methyloxycarbonyl or benzyl.

In the reaction Scheme 2, T and X₁ can for example be independently selected from the group consisting of Cl, Br, I, OTf, OTs, OPiv or T can be a metal species M. Preferably M is a metal species comprising a metal ion selected from the group consisting of Mg, Al, Zn, Zr, B, Sn, Si, more preferably M is ZnCl, ZnBr, ZnI, Zn(Alkyl), B(OH)₂, B(OC(CH₃)₂C(CH₃)₂O), 9-BBN, B(Sia)₂, B(Cat), B(Cy)₂, BF₃⁻ or B(MIDA). Generally, T and X₁ can for example independently be Cl, Br, I, OTf, OTs, OPiv, MgCl, MgBr, MgI, Sn(Alkyl)₃, Si(Alkyl)₃, Si(OAlkyl)₃, ZnCl, ZnBr, ZnI, Zn(Alkyl), B(OH)₂, B(OC(CH₃)₂C(CH₃)₂O), 9-BBN, B(Sia)₂, B(Cat), B(Cy)₂, BF₃⁻, B(MIDA).

The term “Alkyl” herein refers to a radical or part of a radical that is a straight or branched (one or, if desired and possible, more times) carbon chain. It can be C₁-C₈-alkyl. The term “C₁-C₈-” defines a moiety with up to and including maximally 8 carbon atoms, said moiety being branched (one or more times) or straight-chained and bound via a terminal or a non-terminal carbon. C₁-C₈-alkyl, for example, is n-pentyl, n-hexyl or n-heptyl, n-octyl, methyl, ethyl, n-propyl, isopropyl, n-butyl, isobutyl, sec-butyl, tert-butyl, in particular methyl, ethyl, n-propyl, isopropyl, n-butyl, isobutyl, sec-butyl, tert-butyl.

The catalyst used to perform the reaction outlined in Scheme 2 is any catalyst that a skilled person would select based on general textbook. The catalyst can be, for example, selected from the group consisting of Pd(PPh₃)₂Cl₂, Pd(PPh₃)₄, Pd(dba)₂, Pd₂(dba)₃, Pd(OAc)₂, [Pd(allyl)Cl]₂, Pd(dppf)Cl₂, PdBr₂(PtBu₃)₂, PdCl(crotyl)(PtBu₃), Pd(PtBu₃)₂, PdCl₂(Amphos)₂, PdCl(allyl)(Amphos), PdBr₂(Binap), PdCl₂(DCPP), PdCl₂(DiPrPF), PdCl₂(DiPrPF), Pd-PEPPSI-IPr, Chloro(2-dicyclohexylphosphino-2′,4′,6′-triisopropyl-1,1′-biphenyl)[2-(2-aminoethyl)phenyl)]palladium(II) (also known as XPhos Precatalyst 1^{st} Generation), Chloro(2-dicyclohexylphosphino-2′,4′,6′-triisopropyl-1,1′-biphenyl)[2-(2′-amino-1,1′-biphenyl)]palladium(II) (also known as XPhos Precatalyst 2^{nd} Generation), Chloro(2-dicyclohexylphosphino-2′,6′-dimethoxy-1,1′-biphenyl)[2-(2-aminoethylphenyl)]palladium(I) (also known as SPhos Precatalyst 1^{st} Generation), Chloro(2-dicyclohexylphosphino-2′,6′-dimethoxy-1,1′-biphenyl)[2-(2′-amino-1,1′-biphenyl)]palladium(II) (also known as XPhos Precatalyst 2^{nd} Generation), Chloro(2-dicyclohexylphosphino-2′,6′-diisopropoxy-1,1-biphenyl)[2-(2-aminoethylphenyl)]palladium(II) (also known as RuPhos Precatalyst 1^{st} Generation), Chloro(2-dicyclohexylphosphino-2′,6′-diisopropoxy-1,1′-biphenyl)[2-(2′-amino-1,1′-biphenyl)]palladium(II) (also known as RuPhos Precatalyst 2^{nd} Generation), Pd/C, Pd, Ni(acac)₂, NiCl₂, Ni(PPh₃)₂Cl₂, Ni(cod)₂, Ni(dppf)(cod), Ni(dppf)(cinnamyl), Ni(dppf)₂, Ni(dppf)Cl₂, Ni(dppp)Cl₂, NiCl₂(PCy₃)₂, Ni(dppe)Cl₂, or mixtures thereof.

The co-catalyst or additive can be selected from the group consisting of ZnCl₂, ZnBr₂, CuI, LiCl, PPh₃, P(oTol)₃, P(oTol)Ph₂, P(pTol)₃, PtBu₃, PtBu₃*HBF₄, PCy₃, PCy₃*HBF₄, P(OiPr)₃, DPE-Phos, dppf, dppe, dppp, dcpp, dppb, P(Furyl)₃, CPhos, SPhos, RuPhos, XPhos, DavePhos, JohnPhos and Xantphos.

The catalyst can be generally present in an amount up to 10.0 mol %. Typically, the catalyst may be present in an amount below 5.0 mol %. The catalyst can be further present in a range from about 0.005 mol % to about 5.0 mol %, about 0.01 mol % to about 1.0 mol %, or from about 0.05 mol % to about 0.5 mol %, based on the starting compound of formula (A). Typically, the catalyst may be present in an amount of about 0.1 mol %.

The reaction described in Scheme 2 is performed particularly well when T is Br, X₁ is ZnI and P is tert-butyloxycarbonyl (Boc). The preferred solvent for the reaction is tetrahydrofuran (THF), the catalyst is Pd(PPh₃)₂Cl₂ and the co-catalyst or additive is CuI. Particularly the reaction performs well at a temperature over 25° C., preferably about 50° C.

The term “co-catalyst or additive” as used herein refers to a chemical agent that enhances the rate of a chemical reaction by lowering the activation energy. The co-catalyst can be a heterogeneous catalyst or a homogenous catalyst and the additive can be a ligand, a salt or any other chemical species that can enhanced the reactivity of the catalyst.

The term “ligand” means any compound, achiral or chiral, that can form a complex with a transition metal. Chiral and achiral ligands can be, for example, CPhos, SPhos, XPhos, DavePhos, JohnPhos, DPE-Phos and Xantphos.

### 1.2 Alternative Route to Access Compound of Formula (C2-1)

A further aspect of the disclosure relates to a process for preparing a compound of formula (C2-1), comprising the steps of reacting a compound of formula (C2-3) with a pyridinium-P substituent in a solvent to obtain a compound (C2-2); and transforming the compound (C2-2) to obtain the compound of formula (C2-1) as described in Scheme 3. According to the disclosure the protecting group (P) can be a customary protecting group, particularly can be chosen from those mentioned herein (for example tert-butyloxycarbonyl (Boc), benzyloxycarbonyl, methyloxycarbonyl, ethyloxycarbonyl, allyloxycarbonyl, phenyloxycarbonyl, formyl, acetyl or benzyl). Methyloxycarbonyl and Boc are preferred protecting groups used in the process depicted in Scheme 3. Methyloxycarbonyl is the most preferred.

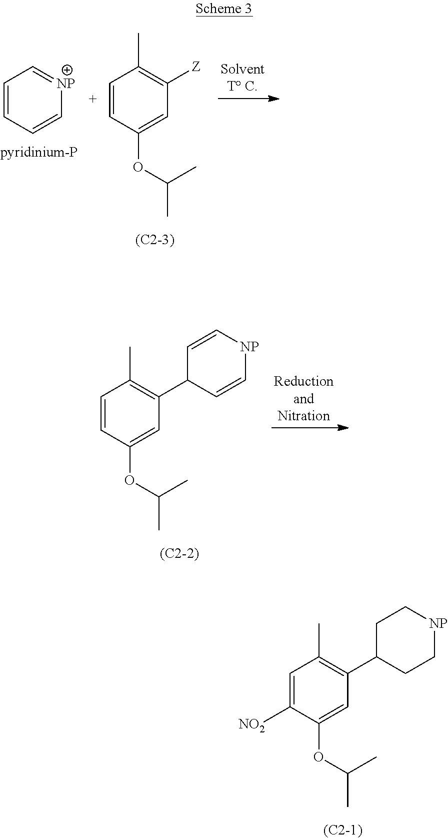

The reaction between the pyridinium-P and the compound of formula (C2-3) is best performed when Z is selected from MgBr, MgI, MgCl, ZnCl, ZnBr, ZnI, Zn(Alkyl) or the like, preferably when Z is MgCl or MgBr, most preferably MgBr.

Suitable solvents for obtaining a compound of formula (C2-2) are, for example, aprotic polar solvents. For example, the reaction can be well steered in tetrahydrofuran (THF), 2-methyl-tetrahydrofuran, acetonitrile, dichloromethan, 1,4-dioxane or diethyl ether, or mixtures thereof. The reaction is particularly well performed in tetrahydrofuran (THF).

The synthesis of compound of formula (C2-2), as described in Scheme 3, is performed particularly well when Z is selected as MgBr and P is selected as methyloxycarbonyl, in tetrahydrofuran (THF), at a temperature between −30° C. to reflux. The temperature may need to be higher than room temperature in order to get good yields if Z is selected from less reactive Mg or Zn species or the like. With particularly reactive Grignard substituents such as Z being MgBr or MgCl, the temperature can be between −30° C. and room temperature.

Compound of formula (C2-2) is then transformed into a compound of formula (C2-1) as described in Scheme 3.

The reduction is performed with hydrogen or a hydrogen donor (transfer hydrogenation) in the presence of a catalyst, with or without an additive. As a hydrogen donor water, acids (e.g. formic acid), alkalis, alcohols, amines, liquid ammonia, ammonium formate or the like can be used.

The catalyst used to perform the reduction of a compound of formula (C2-1) is any catalyst that a skilled person would select based on general textbook. The catalyst can be selected from the group consisting of Raney nickel, Pt/C, Rh/C, Pd/Al₂O₃, Pd/CaCO₃, RhCl(PPh₃)₃, Lindlar catalyst, PtO₂, Pd/C, [Rh(cod)(PPh₃)₂]⁺, [Ir(cod)(PCy₃)(Py)]⁺, Pd(OH)₂, Pd(OAc)₂, Pd₂(dba)₃, Zn, Fe, Sm, NiCl₂, Ni(OAc)₂, CoCl₂, ZrCl₄, TiCl₃. The reaction is performed particularly well with Pd/C. The catalyst can be present in a range from about 0.005 mol % to about 30.0 mol %. Typically, the catalyst may be present in an amount of about 10.0 mol %.

An additive can optionally be added and can be selected from the group of ZnCl₂, ZnBr₂, CuI, LiCl, PPh₃, P(oTol)₃, P(oTol)Ph₂, P(pTol)₃, PtBu₃, PtBu₃*HBF₄, PCy₃, PCy₃*HBF₄, P(OiPr)₃, DPE-Phos, dppf, dppe, dppp, dcpp, dppb, P(Furyl)₃, CPhos, SPhos, RuPhos, XPhos, DavePhos, JohnPhos and Xantphos.

The reduction may be for example performed in a solvent selected from the group consisting of water, methanol, ethanol, propanol, isopropanol, 1-butanol, 2-butanol, tertbutanol, acetic acid, tetrahydrofuran (THF), dichloromethane, diethyl ether, tertbutyl methyl ether, ethyl acetate, toluene, 1,4-dioxane, acetonitrile or acetone, or mixtures thereof.

The nitration of the benzene ring, as mentioned in Scheme 3, is any nitration reaction that a skilled person would select based on general textbook. The source of “NO₂” can be selected from HNO₃ or a salt thereof. Suitable solvents for the transformation can be for example selected from acetic anhydride, sulfuric acid or trifluoroacetic acid.

The reduction, as described in Scheme 3, is performed particularly well with 10% Pd/C, ammonium formate, in methanol and tetrahydrofuran (THF) at room temperature. The nitration is performed particularly well in the presence of HNO₃ and acetic anhydride. The nitro group can be introduced to the benzene ring at a temperature below 0° C., particularly around −10° C.

### 1.3 Compound of Formula (C2):

A compound of formula (C2), or a salt thereof, can be prepared according to the process summarized in Scheme 4. The process involves reducing and deprotecting a compound of formula (C2-1) as disclosed herein to prepare a compound of formula (C2), or a salt thereof.

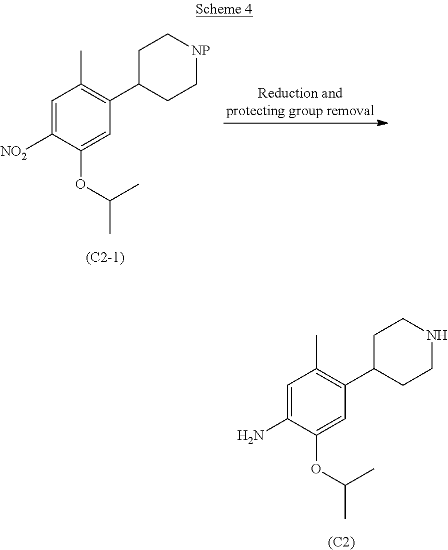

In one embodiment the reduction of compound (C2-1) that possesses a protecting group P as described for Scheme 2, is performed with hydrogen in the presence of a catalyst. The protecting group P refers to a nitrogen protecting group selected from the above mentioned list (for example tert-butyloxycarbonyl (Boc), benzyloxycarbonyl, methyloxycarbonyl, ethyloxycarbonyl, allyloxycarbonyl, phenyloxycarbonyl, formyl, acetyl or benzyl).

The catalyst used to perform the reduction of compound of formula (C2-1) is any catalyst that a skilled person would select from a general textbook. The catalyst can be selected from the group consisting of Raney nickel, Pt/C, Rh/C, Pd/Al₂O₃, Pd/CaCO₃, RhCl(PPh₃)₃, Lindlar catalyst, PtO₂, Pd/C, [Rh(cod)(PPh₃)₂]⁺, [Ir(cod)(PCy₃)(Py)]⁺, Pd(OH)₂, Pd(OAc)₂, Pd₂(dba)₃, Zn, Fe, Sm, NiCl₂, Ni(OAc)₂, CoCl₂, ZrCl₄, TiCl₃. The catalyst can be present in a range from about 0.005 mol % to about 20.0 mol %. Typically, the catalyst can be present in an amount below 10.0 mol % (to about 0.005 mol %).

The term “catalyst” as used herein refers to a catalytic amount of a chemical agent that enhances the rate of a chemical reaction by lowering the activation energy for the chemical reaction. The catalyst can be a heterogeneous catalyst or a homogenous catalyst.

The term “heterogeneous catalyst” refers to a catalyst supported on a carrier, typically although not necessarily a substrate comprised of an inorganic material, for example, a porous material such as carbon, silicon and/or aluminum oxide.

The term “homogeneous catalyst” refers to a catalyst that is not supported on a carrier.

The terms “hydrogen” or “hydrogenation” used to describe a chemical reaction refer to the action of reducing another compound in the presence of hydrogen. The source of hydrogen can be selected from gaseous hydrogen (H₂), hydrogen donors (transfer hydrogenation, e.g. formic acid or salts thereof), hydride reagent (BH₃, B₂H₆. NaBH₄) or the like.

The reduction may be performed in a solvent such as an alcohol based solution. The alcohol based solution can comprise or consist of C₁ to C₁₀ alcohols (e.g. methanol, ethanol, propanol, isopropanol and butanol) or mixtures thereof.

The reaction of reduction is best carried out at elevated pressure, preferably between 1 bar and 10 bar, particularly at 4 bar. Reduction of the compound of formula (C2-1), or a salt thereof, is best done by stirring the reaction mixture for 5 hours at room temperature, with 10 mol % of Pd/C and hydrogen in the presence of ethanol. The reduction is best performed with the protecting group P being selected from tert-butyloxycarbonyl (Boc) or methyloxycarbonyl.

The term “room temperature” or “ambient temperature” as used herein, unless specified otherwise, means a temperature from 15 to 30° C., such as from 20 to 30° C., particularly such as from 20 to 25° C.

In addition to reducing the compound (C2-1) the protecting group gets cleaved off. The removal of the protecting group can be carried out under standard reaction conditions known in the art. Unless otherwise specified, the protecting group can be removed in the absence or, customarily, in the presence of acids or bases, preferably acids or bases that cause removal of the protecting group but at the same time do not cause chemical degradation of the compounds and intermediates. The removal of the protecting group can also be carried out under reductive conditions, e.g. during the first nitro reduction step of (C2-1). Preferably, the protecting group is removed with an acid. Particularly suitable acids for the removal of the protecting group P are HF.pyridine, HF.triethylamine ammonium fluoride, hexafluoroisopropanol, acetic acid, trifluoroacetic acid, hydrochloric acid, sulfuric acid, or a combination thereof. Preferably the acid is trifluoroacetic acid or hydrochloric acid. The reaction consisting of removing the protecting group can take place in a solvent that facilitates the removal of the protecting group. As an example, the protecting group can be removed in a solvent selected from the group consisting of dichloromethane, ethyl acetate, 1,4-dioxane, diethyl ether, tetrahydrofuran (THF), methanol or acetonitrile. The protecting group is removed by stirring the reaction mixture for at least 8 hours, preferably for 16 hours at a temperature between −78° C. and 70° C., preferably between 0° C. and 70° C. The deprotection reaction for the tert-butyloxycarbonyl (Boc) protecting group is performed best with trifluoroacetic acid in dichloromethane, optionally at ambient temperature. The deprotection reaction for the methyloxycarbonyl protecting group is performed best with hydrochloric acid, optionally at 60° C.

In one embodiment, reduction and protecting group removal are performed simultaneously.

### 1.4 Alternative Route to Prepare the Compound of Formula (C2):

The compound of formula (C2), or a salt thereof, can be prepared by an alternative process. Namely, a compound of formula (C) is reduced in a solvent in the presence of at least one catalyst and hydrogen, as depicted in Scheme 5.

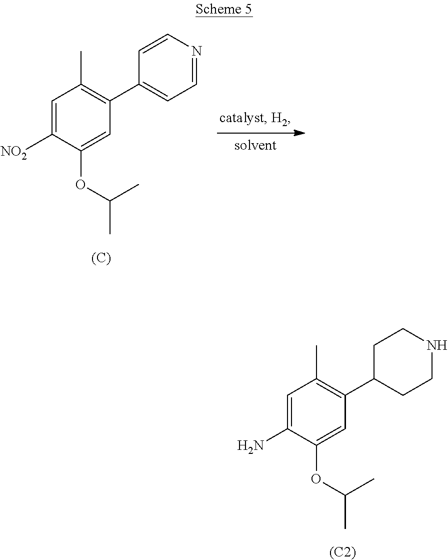

The reaction for the preparation of the compound of formula (C2), or a salt thereof, can be carried out as a one-pot process by reducing two functional groups present on compound of formula (C) simultaneously to allow a rapid access to a compound of formula (C2), or a salt thereof. The reduction step involves the presence of a catalyst and hydrogen in a solvent.

The catalyst used to perform the reduction of compound of formula (C) is any catalyst that a skilled person would select from a general textbook. The catalyst for example can be selected from the group consisting of Raney nickel, Pt/C, Rh/C, Pd/Al₂O₃, Pd/CaCO₃, RhCl(PPh₃)₃, Lindlar catalyst, PtO₂, Pd/C, [Rh(cod)(PPh₃)₂]⁺, [Ir(cod)(PCy₃)(Py)]⁺, Pd(OH)₂, Pd/Al, Pt/Al, Pt/SiAl and Pd/ZrO₂, or mixtures thereof.

The catalyst can be present in a range from about 0.005 mol % to about 50.0% w/w (dry) based on the starting compound of formula (C). Typically, the catalyst may be present in an amount below 20.0% w/w (dry).

The reduction of the compound of formula (C) is done by stirring the reaction mixture for up to 16 hours, preferably between 8 and 16 hours at a temperature between 10 and 40° C., preferably between 20 and 30° C. The reaction is best carried out at a pressure between 1 and 15 bar, preferably between 2 and 6 bar.

The solvent used in the reaction can be for example selected from the group consisting of water, methanol, ethanol, propanol, isopropanol, 1-butanol, 2-butanol, tertbutanol, acetic acid, tetrahydrofuran (THF), dichloromethane, diethyl ether, tertbutyl methyl ether, ethyl acetate, toluene, benzene, 1,4-dioxane, acetonitrile and acetone, or mixtures thereof.

The process of reducing the compound of formula (C) to a compound of formula (C2), or a salt thereof, is performed particularly well in acetic acid in the presence of Pd/Al and hydrogen. Reaction conditions are preferably set to about 60° C. and a pressure of about 80 bar. The reaction is also performed well in the presence of Pt/C and hydrogen, optionally in acetic acid. In this case the temperature is best if set at about 10 to about 50° C. and the pressure at about 1 to about 10 bar.

### 1.5 Compound of Formula (C):

Compound (C) can be prepared as shown in Scheme 6 in an one-pot process by reacting in a solvent a compound of formula (AA) with a compound of formula (D), in the presence of X₃B(X₂)₂, a base, a catalyst, and optionally a ligand, wherein:

Y is selected from Cl, Br, I, OTf, OTs, OPiv and OMs, preferably Cl, Br;
X₄ is selected from Cl, Br, I, OTf, OTs, OPiv and OMs, preferably Cl, Br;
B(X₂)₂ is selected from B(OH)₂, B(OC(CH₃)₂C(CH₃)₂O), 9-BBN, B(Sia)₂, B(Cat), B(Cy)₂;
X₃ is H, B(X₂)₂.

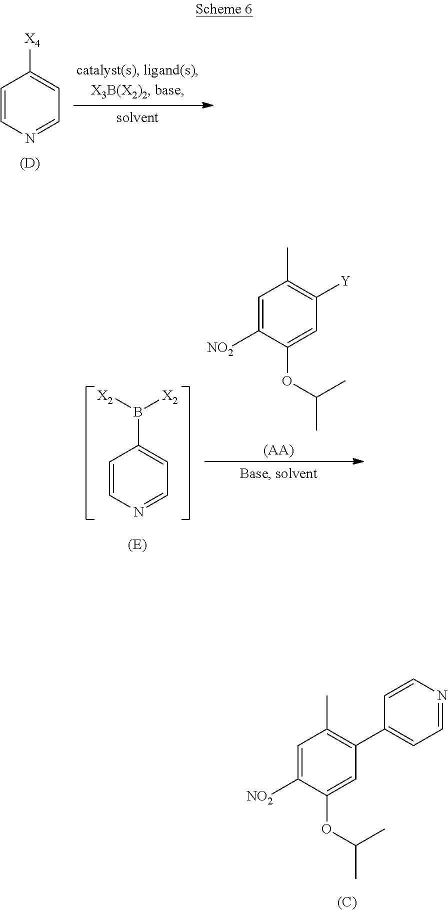

One embodiment of the disclosure provides an in-situ formation of a compound of formula (E), without isolation of a compound of formula (E), in a solvent in the presence of a catalyst, X₃B(X₂)₂, a base and optionally a ligand. The reaction can be one-pot reaction. The process of reacting the compounds of formula (AA) and formula (D) is done by first stirring the reaction mixture containing compound of formula (D) and the catalyst for about 1 to 10 hours, preferably for 3 hours at a temperature between 40° C. and reflux temperature, preferably at 100° C. Then compound of formula (AA) is added and the reaction mixture is stirred for about 1 to 48 hours, preferably for about 17 hours at a temperature between 40° C. and reflux temperature, preferably at about 100° C.

The catalyst used in the one-pot reaction is any catalyst that a skilled person would select from a general textbook. The catalyst can include a ligand and can for example be selected from the group consisting of Pd(PPh₃)₂Cl₂, Pd(PPh₃)₄, Pd(dba)₂, Pd₂(dba)₃, Pd(OAc)₂, [Pd(allyl)Cl]₂, Pd(dppf)Cl₂, PdBr₂(PtBu₃)₂, PdCl(crotyl)(PtBu₃), Pd(PtBu₃)₂, PdCl₂(Amphos)₂, PdCl(allyl)(Amphos), PdBr₂(Binap), PdCl₂(dcpp), PdCl₂(DiPrPF), PdCl₂(DiPrPF), Pd-PEPPSI-IPr, Chloro(2-dicyclohexylphosphino-2′,4′,6′-triisopropyl-1,1′-biphenyl)[2-(2-aminoethyl)phenyl)]palladium(II), Chloro(2-dicyclohexylphosphino-2′,4′,6′-triisopropyl-1,1′-biphenyl)[2-(2′-amino-1,1′-biphenyl)]palladium(II), Chloro(2-dicyclohexylphosphino-2′,6′-dimethoxy-1,1′-biphenyl)[2-(2-aminoethylphenyl)]palladium(II), Chloro(2-dicyclohexylphosphino-2′,6′-dimethoxy-1,1′-biphenyl)[2-(2′-amino-1,1′-biphenyl)]palladium(II), Chloro(2-dicyclohexylphosphino-2′,6′-diisopropoxy-1,1-biphenyl)[2-(2-aminoethylphenyl)]palladium(II), Chloro(2-dicyclohexylphosphino-2′,6′-diisopropoxy-1,1′-biphenyl)[2-(2′-amino-1,1′-biphenyl)]palladium(I), Pd/C, Pd, Ni(acac)₂, NiCl₂, Ni(PPh₃)₂Cl₂, Ni(cod)₂, Ni(dppf)(cod), Ni(dppf)(cinnamyl), Ni(dppf)₂, Ni(dppf)Cl₂, Ni(dppp)Cl₂, NiCl₂(PCy₃)₂ and Ni(dppe)Cl₂. The catalyst can be present in an amount up to 10.0 mol %. Typically, the catalyst may be present in an amount below 6.0 mol %.

The ligand used to perform the one-pot reaction is any ligand that a skilled person would select based on general textbook. The ligand can be selected from the group consisting of PPh₃, P(oTol)₃, P(oTol)Ph₂, P(pTol)₃, PtBu₃, PtBu₃*HBF₄, PCy₃, PCy₃*HBF₄, P(OiPr)₃, DPE-Phos, dppf, dppe, dppp, dcpp, dppb, P(Furyl)₃, CPhos, SPhos, RuPhos, XPhos, DavePhos, JohnPhos and Xantphos. The ligand can be present in a range from about 0.005 mol % to about 20 mol %. Typically, the ligand may be present in an amount of below 10 mol %.

The reaction can be performed in a solvent selected for example from 1,4-dioxane, tetrahydrofuran (THF), 2-methyl tetrahydrofuran, diethyl ether, toluene, dimethylformamide (DMF), dimethylacetamide (DMA), dimethylsulfoxide (DMSO), dimethoxyethane (DME), dichloromethane, N-methyl-2-pyrrolidone (NMP), 1-butyl-2-pyrrolidone (NBP), acetonitrile, acetone, dimethylcarbonate, ethylacetate, isopropylacetate, tertbutylacetate, pentane, hexane, heptane, anisole, pyridine, triethylamine, water, methanol, ethanol, n-propanol, 2-propanol, n-butanol, 2-butanol, tert-butanol, or mixtures thereof.

The base used to perform the reaction is any base that a skilled person would select based on a general textbooks. The base can be for example Na₂CO₃, K₂CO₃, Cs₂CO₃, Tl₂CO₃, NaHCO₃, KHCO₃, NaOAc, KOAc, Na₃PO₄, K₃PO₄, LiOH, NaOH, KOH, CsOH, Ba(OH)₂, NaOMe, KOMe, NaOEt, KOEt, TlOEt, NaOPh, NEt₃, DIPEA, NaOtBu, KOtBu, KF or CsF.

The substituent of the boronyl group or X₃B(X₂)₂ may form together with a boron a group of formula —B(X₂)₂, wherein the two X₂ substituents are the same or different and can be halogen, hydroxy, C₁-C₄ alkoxy, or the two X₂ substituents together form a residue of a diol. Preferably, the boronyl group thereof of the formula —B(X₂)₂ may be a group of the formula —B(OR′)OR″, wherein R′ and R″, independently of one another, are identical or different and each can be hydrogen or C1-C₁₂-alkyl, and where R and R may be bridged in a cyclic manner, for example, R and R′ combined are alkylene which together with the boron and the oxygen atoms form a 5- or 6-membered ring. The boron derivative used to perform the reaction is any organoboron derivatives that a skilled person would select based on a general textbook. The organoboron can be selected for example from the group consisting of bis(pinacolato)diboron, tetrahydroxydiboron, pinacolborane and neopentylglycolborane.

The reaction of a compound of formula (D) with the organoboron will engender a compound of formula (E) that possesses a —B(X₂)₂ group selected from the group consisting of —B(OH)₂, —B(OC(CH₃)₂C(CH₃)₂O).

The process of generating in-situ a compound of formula (E) by reacting a compound of formula (D) is performed particularly well in 1,4-dioxane in the presence of bis(pinacolato)diboron, X-Phos, Chloro(2-dicyclohexylphosphino-2′,4′,6′-triisopropyl-1,1′-biphenyl)[2-(2′-amino-1,1′-biphenyl)]palladium (II) and KOAc. The temperature can be set at 100° C. Then the compound of formula (E) generated in-situ is reacted with a compound of formula (AA) in the presence of a base in a solvent. The reaction is best carried out at a temperature between 40° C. and reflux, preferably at 100° C. Water and K₂CO₃ are added to the reaction mixture to initiate the coupling reaction between the newly formed compound (E) and the compound of formula (AA). The reaction can continue to run at 100° C.

The term “one pot process” refers to the fact that the relevant step is performed in sequence without isolating the product of each step. Furthermore the process is additionally simplified by omitting removal or exchange of any other component of the reaction mixture. It also increases occupational safety by omitting the need for the isolation of potential toxic or hazardous intermediates. Using a one pot process is a simple and cost effective method of organic synthesis but is only commercially valuable where the level of impurities can be minimized to give a reasonable yield. In the present case, the described one-pot process yields good results and an acceptable level of unreacted intermediates and side products.

In alternative, the compound of formula (C), or a salt thereof, can be also prepared by Suzuki-Coupling of compounds of formula (AA) and isolated compound of formula (E). The reaction can be performed in a solvent in the presence of a catalyst and a base. In formula (AA) and (E), Y denotes Cl, Br, I, OTf, OTs, OPiv and OMs; and B(X₂)₂ denotes B(OH)₂, B(OC(CH₃)₂C(CH₃)₂O), 9-BBN, B(Sia)₂, B(cat), B(Cy)₂, BF₃⁻ and B(MIDA), respectively. In a particular embodiment, Y is Cl and X₂ is OH. The solvent used can be for example water, methanol, ethanol, n-propanol, 2-propanol, n-butanol, 2-butanol, dimethylformamide (DMF), tetrahydrofuran, 2-methyl tetrahydrofuran, toluene, dioxane, or mixtures thereof. Preferably, the solvent is 2-butanol and water. The same catalyst and base as above can be selected. In one embodiment, the catalyst is Pd(PPh₃)₂Cl₂. The preferred base is K₂CO₃.

In one embodiment, the compound of formula (AA) is reacted with the compound of formula (E) in 2-butanol or water in the presence of Pd(PPh₃)₂Cl₂ as a catalyst and K₂CO₃ as a base, wherein Y is Cl and X₂ is OH.

### 1.6 Compound of Formula (C3-1):

Another useful intermediate in preparation of ceritinib is the compound of formula (C3-1),

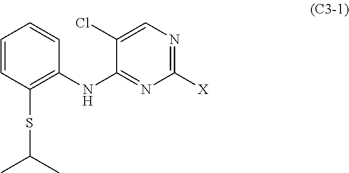

wherein X is selected from halogen (F, Cl, Br, I), alkoxy (preferably OMe, OEt, OtBu), aryloxy (preferably OPh), alkylthio (preferably SMe, SEt), arylthio (SCH₂Ph), sulfinyl (preferably SOMe, SOEt, SOCH₂Ph), sulfonyl (preferably SO₂Me, SO₂Et, SO₂CH₂Ph). Most preferably X is Cl.

The term “alkoxy”, being a radical or part of a radical, refers to alkyl-O—, wherein the term alkyl is as defined herein, and includes, for example, C₁-C₂₀-alkoxy (—O—C₁-C₂₀-alkyl), preferably C₁-C₇-alkoxy (—O—C₁-C₇-alkyl). In particular, alkoxy includes, for example, methoxy (OMe), ethoxy (OEt), n-propyloxy, isopropyloxy, n-butyloxy, isobutyloxy, sec-butyloxy, tert-butyloxy (OtBu), pentyloxy, hexyloxy and heptyloxy radicals. The preferred alkoxy substitutents are methoxy (OMe), ethoxy (OEt) or tert-butyloxy (OtBu).

The term “aryl”, being a radical or part of a radical, refers to an aromatic hydrocarbon group, for example, C₆-C₁₀-aryl, and is preferably a mono- or polycyclic, especially monocyclic, bicyclic or tricyclic aryl moiety with 6 to 14 carbon atoms, for example 6 to 10 carbon atoms. Preferably aryl denotes phenyl, benzyl, indenyl, indanyl or naphthyl. The term “Aryloxy” refers to an Aryl-O—, wherein aryl is as defined above. In particular, the preferred aryloxy herein is phenoxy (OPh). The term “aryllthio” refers to Aryl-S—, wherein aryl is as defined above. The preferred arylthio is benzylthio (SCH₂Ph).

The term “alkylthio” refers to alkyl-S—, wherein alkyl is as defined herein. The alkyl group for example comprises 1 to 8 carbon atoms. In particular, alkylthio includes, for example, methylthio (SMe), ethylthio (SEt), phenylthio (PhS) and pentylthio. The preferred alkylthio substituents herein are methylthio (SMe) and ethylthio (SEt).

The term “sulfinyl” corresponds to a —S—O-alkyl group that includes C₁-C₈-alkyl linear or branched. In particular, sulfinyl includes, for example, methylsulfinyl (SOMe), ethylsulfinyl (SOEt), phenylsulfinyl (SOPh) and benzylsulfinyl (SOCH₂Ph).

The term “sulfonyl” refers to the divalent —S(O)₂— group. In particular, sulfonyl includes, for example methylsulfonyl, ethylsulfonyl, phenylsulfonyl and benzylsulfonyl.

The compound of formula (C3-1) can be prepared as depicted in Scheme 7 by reacting a compound of formula (F), or a salt thereof, with a compound of formula (G), optionally in the presence of a base, optionally in a solvent. X is selected from the group consisting of halogens (F, Cl, Br, I), alkoxy (preferably OMe, OEt, OtBu) and Aryloxy (preferably OPh), most preferably X is Cl.

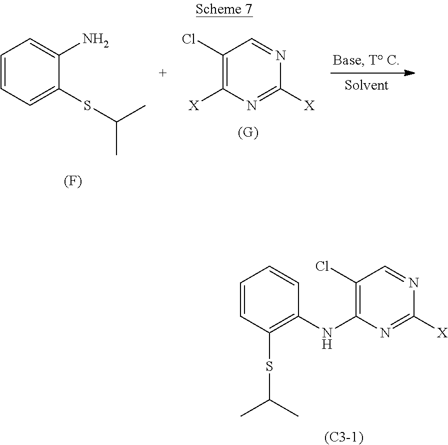

The base used to perform the reaction is any base that a skilled person would select based on a textbook. It can be selected from the group consisting of Na₂CO₃, K₂CO₃, Cs₂CO₃, NaHCO₃, KHCO₃, triethylamine, DIPEA, Na₃PO₄, K₃PO₄, DBU and NaH. Preferably, the base is DBU or DIPEA. Use of mild base in the reaction results in good efficacy; the base is easy and safe to handle, and potentially prevents side reactions and thus allows recovery of compound of formula (C3-1) in high yield.

The reaction may be performed in a protic or aprotic solvent. For example, the solvent can be selected from the group consisting of 1,4-dioxane, tetrahydrofuran (THF), 2-methyl tetrahydrofuran, diethyl ether, toluene, N-methyl-2-pyrrolidone (NMP), 1-butyl-2-pyrrolidone (NBP), acetonitrile, acetone, dimethylcarbonate, ethylacetate, isopropylacetate, tertbutylacetate, water, methanol, ethanol, n-propanol, 2-propanol, n-butanol, 2-butanol, tert-butanol and toluene, or mixtures thereof. Alternatively, the solvent can also be omitted.

The compound of formula (F), or a salt thereof, and the compound of formula (G) can be reacted by stirring the reaction mixture for 1 hour to 72 hours, preferably for about 18 hours. Temperature between 40° C. and reflux temperature can be chosen, but the temperature is preferably between 100° C. and 115° C., particularly is about 110° C.

The process of reacting a compound of formula (F), or a salt thereof, and a compound of formula (G) is best carried out in toluene and 1-butanol in the presence of DIPEA. Temperature is best set at 100-115° C. Such reaction can be stirred for about 18 hours.

In one embodiment, X in compounds of formula (G) and (C3-1) denotes Cl. A particular embodiment of the present disclosure is the compound of formula (C3-1), wherein X is Cl.

The compound of formula (C3-1) can be used for preparing ceritinib.

### 1.7 Compound of Formula (C3):

From the compound of formula (C3-1), a compound of formula (C3) can be prepared. As shown in Scheme 8, the compound (C3) can be prepared by oxidizing the obtained compound of formula (C3-1), wherein X is selected from halogen (F, Cl, Br, I), alkoxy (preferably OMe, OEt, OtBu) and aryloxy (preferably OPh), sulfinyl (preferably SOMe, SOEt, SOCH₂Ph), sulfonyl (preferably SO₂Me, SO₂Et, SO₂CH₂Ph), most preferably X is Cl.

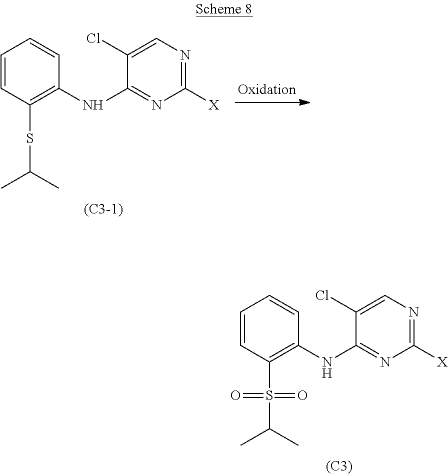

The oxidation can be performed in water or an organic solvent. The solvent can be selected from 1,4-dioxane, tetrahydrofuran (THF), 2-methyl tetrahydrofuran, diethyl ether, toluene, dimethylformamide (DMF), dimethylacetamide (DMA), dimethylsulfoxide (DMSO), dimethoxyethane (DME), dichloromethane, N-methyl-2-pyrrolidone (NMP), 1-butyl-2-pyrrolidone (NBP), acetonitrile, acetone, dimethylcarbonate, ethyl acetate, isopropylacetate, tertbutylacetate, pentane, hexane, heptane, anisole, pyridine, triethylamine, acetic acid, water, methanol, ethanol, n-propanol, 2-propanol, n-butanol, 2-butanol and tert-butanol, or mixtures thereof.

The oxidative reagent can be selected, among others, from the group consisting of KMnO₄, MnO₂, NaIO₄, NaClO, KHSO₅ (Oxone), NaBO₃, CH₃CO₃H, H₂O₂, Na₂WO₄, O₂, O₃, tetrapropylammonium perruthenate (TPAP), 3,3-dimethyldioxirane, 3-chloroperoxybenzoic acid (mCPBA) and tertbutylhydroperoxide (TBHP), or mixtures thereof, optionally with a catalyst.

Preparing the compound of formula (C3) from the compound of formula (C3-1) can include stirring the reaction mixture for 4 to 40 hours, preferably for about 16 hours at a temperature between 10 and 60° C., preferably between 20 and 40° C., particularly at about 30° C. The oxidation of the compound of formula (C3-1) can be carried out in high yield in ethyl acetate, in the presence of CH₃CO₃H as a solution in CH₃CO₂H at 30° C. or the process can be carried out in high yield in methanol, in the presence of H₂O₂ and Na₂WO₄ at the temperature between 20-70° C.

### 1.7 Alternative Route to Prepare Compound of Formula (C3)

The compound of formula (C3) can alternatively be prepared by following the steps of (i) reacting a compound of formula (H) with a compound of formula (I) to obtain an intermediate; (ii) reducing the intermediate to form compound (J); and (iii) reacting the compound (J) with a compound of formula (G) in the presence of a base, as described in Scheme 9, vide infra.

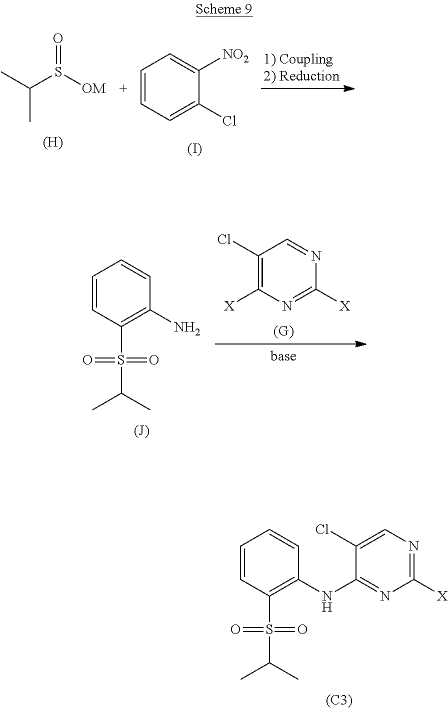

X can denote, as above, F, Cl, Br, I, Alkoxy (preferably OMe, OEt, OtBu), Aryloxy (preferably OPh), alkylthio (preferably SMe, SEt), arylthio (preferably SCH₂Ph), sulfinyl (preferably SOMe, SOEt, SOCH₂Ph), sulfonyl (preferably SO₂Me, SO₂CH₂Ph). Most preferably X is Cl. M can be selected from Li, Na, K, 0.5 Zn, 0.5 Ca, preferably M is Na.

Compound of formula (H) and the compound of formula (I) react in a solvent while being stirred at a temperature between the room temperature and reflux. In specific embodiment the reaction is conducted in a solvent at a temperature between 82-86° C.

The solvent used for the reaction can be for example dimethylsulfoxide (DMSO), 1,4-dioxane, tetrahydrofuran (THF), 2-methyl tetrahydrofuran, diethyl ether, toluene, dimethylformamide (DMF), dimethylacetamide (DMA), dimethoxyethane (DME), dichloromethane, N-methyl-2-pyrrolidone (NMP), 1-butyl-2-pyrrolidone (NBP), acetonitrile, acetone, dimethylcarbonate, ethyl acetate, isopropylacetate, tertbutylacetate, pentane, hexane, heptane, anisole, pyridine, triethylamine, acetic acid, water, methanol, ethanol, n-propanol, 2-propanol, n-butanol, 2-butanol, tert-butanol, or mixtures thereof. Preferably DMSO is selected.

The reductive step to obtain a compound of formula (J) can include use of a catalyst and hydrogen in a solvent. The catalyst used to perform the reduction is any catalyst that a skilled person would know to select from a general textbook. The catalyst can be for example Raney nickel, Pt/C, Rh/C, Pd/Al₂O₃, Pd/CaCO₃, RhCl(PPh₃)₃, Lindlar catalyst, PtO₂, Pd/C, [Rh(cod)(PPh₃)₂]⁺, [Ir(cod)(PCy₃)(Py)]⁺, Pd(OH)₂, Pd/Al, Pt/Al, Pt/SiAl, Pd/ZrO₂, or mixtures thereof.

The catalyst added to the reaction mixture can be present in a range from about 0.005 mol % to about 50.0% w/w (dry) based on the starting compound of formula (I). Typically, the catalyst may be present in an amount below 20.0% w/w (dry).

The reduction reaction can be stirred for several hours, normally at a temperature up to 60° C., preferably about 40° C.

The reaction of reduction is best carried out at elevated pressure, for example pressure between 1 and 15 bar, preferably between 2 and 6 bar.

The reduction may be performed in a solvent such as an alcohol based solution. The alcohol based solution can be a C₁ to C₁₀ alcohols (e.g. methanol, ethanol, propanol, isopropanol and butanol) or mixtures thereof may be used as the reaction medium. Preferably the solvent is ethanol.

The compound of formula (C3) is obtained by reacting the intermediate of formula (J) with a compound of formula (G) in the presence of a base and in the absence of a solvent. Performing the reaction without a solvent is a really attractive alternative as it is more efficient, engender less waste and reduces the overall cost of the synthesis.

The base used to perform the reaction is any base that a skilled person would select based on a general textbook. The base can be for example selected from the group consisting of Na₂CO₃, K₂CO₃, Cs₂CO₃, NaHCO₃, KHCO₃, triethylamine, DIPEA, Na₃PO₄, K₃PO₄, DBU and NaH. Best conditions are achieved when DBU or DIPEA are used, particularly DBU. The mild base defined herein allows to achieve an efficient process, is easy and safe to handle, and potentially prevents side reactions and thus allows recovery of compound of formula (C3) in high yield and purity.

The reaction between intermediate (J) and compound of formula (G) is best performed at a temperature between 40° C. and reflux, particularly at 80° C.

### 1.8 Ceritinib:

Aforementioned processes can be extended to prepare ceritinib or a salt thereof. Depending on the starting materials and the selected route, a skilled person would know how to combine them to produce building blocks to eventually form ceritinib. Certain variants or alternative processes are described herein below. For example, as shown in Scheme 10, ceritinib, or a salt thereof, can be prepared in a process, the process comprising the steps of:
i. preparing a compound of formula (C2-1) as described in 1.1 or 1.2
ii. preparing a compound of formula (C2), or a salt thereof,
iii. providing a compound (C3), and
iv. reacting a compound of formula (C2) with a compound of formula (C3), to obtain ceritinib, or a salt thereof.

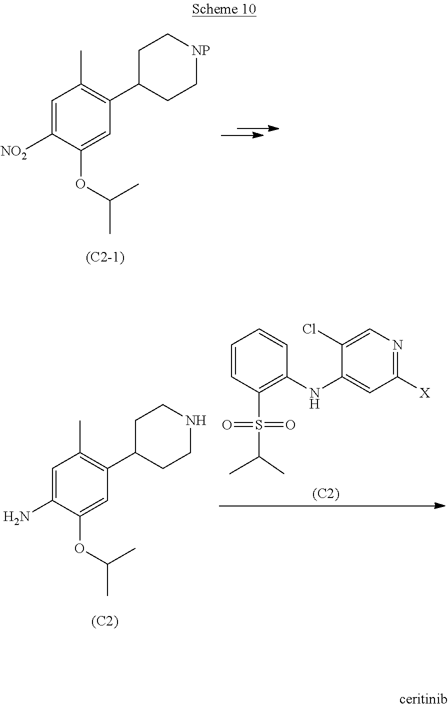

In alternative, ceritinib, or a salt thereof, can be prepared in a process as mentioned in Scheme 11 by:
(a) preparing a compound of formula (C2), or a salt thereof, as described in 1.3 or 1.4
(b) providing a compound of formula (C3), and
(c) reacting the compound of formula (C2), or a salt thereof, with a compound of formula (C3), to obtain ceritinib, or a salt thereof.

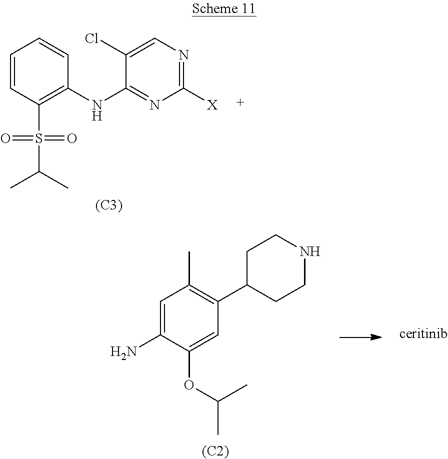

As depicted in Scheme 12, ceritinib, or a salt thereof, can also be produces in a process that comprises:
(aa) providing a compound of formula (C2), or a salt thereof,
(bb) preparing a compound of formula (C3-1) as described in 1.6
(cc) preparing from the compound of formula (C3-1) the compound of formula (C3), and
(dd) reacting the compound of formula (C2), or a salt thereof, with the compound of formula (C3), to obtain ceritinib, or a salt thereof.

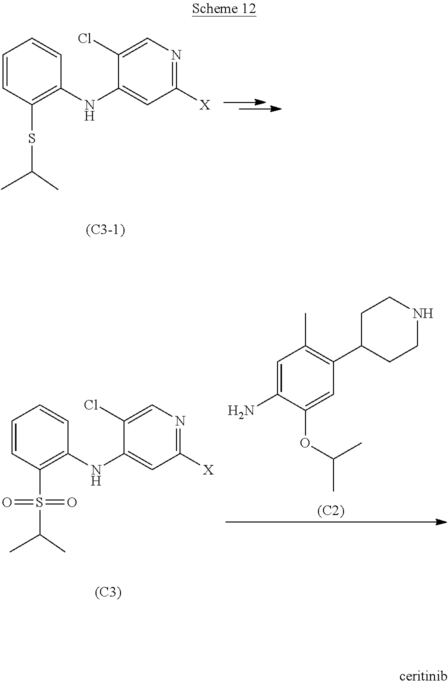

Another variant of the present disclosure relates to a process for preparing ceritinib, or a salt thereof, as mentioned in Scheme 13, comprising
(I) preparing a compound of formula (C) as described in 1.5
(II) preparing from the compound of formula (C) the compound of formula (C2), or a salt thereof,
(III) providing a compound of formula (C3), and
(IV) reacting the compound of formula (C2), or a salt thereof, with the compound of formula (C3), to obtain ceritinib, or a salt thereof.

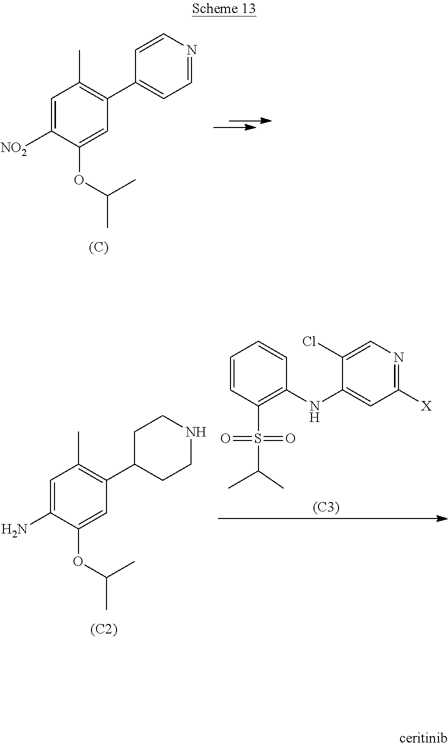

The compound of formula (C2), or a salt thereof, and the compound of formula (C3) can be coupled in a solvent in the presence of a base, wherein X can be halogen (Br, Cl, I), alkoxy (preferably OMe, OEt, OtBu) and aryloxy (preferably OPh), sulfinyl (preferably SOMe, SOEt, SOCH₂Ph) or sulfonyl (preferably SO₂Me, SO₂Et, SO₂CH₂Ph); most preferably X is Cl.

The base for the reaction can be selected from a group of mild bases such as Na₂CO₃, K₂CO₃, Cs₂CO₃, NaHCO₃, KHCO₃, triethylamine, DIPEA, DBU, Na₃PO₄ or K₃PO₄. Alternatively, the base can be omitted.

The reaction can be controlled in vast variety of solvents, for example 1,4-dioxane, tetrahydrofuran (THF), 2-methyl tetrahydrofuran, diethyl ether, toluene, N-methyl-2-pyrrolidone (NMP), 1-butyl-2-pyrrolidone (NBP), acetonitrile, acetone, dimethylcarbonate, ethylacetate, isopropylacetate, tertbutylacetate, water, methanol, ethanol, n-propanol, isopropanol, n-butanol, 2-butanol, tert-butanol and toluene, or mixtures thereof. Preferably tetrahydrofuran (THF), 2-methyl tetrahydrofuran, water, methanol, ethanol, n-propanol, isopropanol, n-butanol, 2-butanol are used, most preferably isopropanol.

The process of reacting the compound of formula (C2), or a salt thereof, to yield the compound of formula (C3) is done by stirring the reaction mixture for 6 to 41 hours, preferably for about 16 hours at a temperature between 40° C. and reflux temperature, preferably at reflux.

The process of reacting a compound of formula (C2) and a compound of formula (C3) is best carried out in isopropanol, at reflux, without a base, hence preventing side reactions and allowing the recovery of ceritinib, or a salt thereof, in high yield and purity.

When salts are referred to herein, it is meant especially pharmaceutically acceptable salts or other generally acceptable salts, unless they would be excluded for chemical reasons, which the skilled person will readily understand. Salts can be formed with final products or intermediates where salt forming groups, such as basic or acidic groups, are present that can exist in dissociated form at least partially, e.g. in a pH range from 4 to 10 in aqueous solutions, or can be isolated especially in solid, especially crystalline, form.

Such salts are formed, for example, as acid addition salts, preferably with organic or inorganic acids, from compounds or any of the intermediates mentioned herein with a basic nitrogen atom (e.g. imino or amino), especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, lactic acid, fumaric acid, succinic acid, citric acid, amino acids, such as glutamic acid or aspartic acid, maleic acid, hydroxymaleic acid, methylmaleic acid, benzoic acid, methane- or ethane-sulfonic acid, ethane-1,2-disulfonic acid, benzenesulfonic acid, 2-naphthalenesulfonic acid, 1,5-naphthalene-disulfonic acid, N-cyclohexylsulfamic acid, N-methyl-, N-ethyl- or N-propyl-sulfamic acid, or other organic protonic acids, such as ascorbic acid.

Ceritinib prepared as described above may optionally be further purified by recrystallisation from a suitable solvent and may optionally be milled or sieved in order to obtain the final pharmaceutically active ingredient.

Once the pharmaceutically active ingredient ceritinib is obtained (for example as described in Section 1.8 above) it can be mixed with a pharmaceutically acceptable excipient. This can be achieved by mixing, granulating, compacting and the like. This way, a pharmaceutical composition can be prepared and used for the preparation of final dosage forms, such as tablets or capsules.

As used in this specification and the appended claims, the singular forms “a”, “an”, and “the” include plural referents unless the context clearly indicates otherwise.

Similarly, “comprise”, “comprises”, “comprising”, “include”, “includes” and “including” are interchangeable and not intended to be limiting.

### Abbreviations

δ Chemical shift
¹³C-NMR Carbon nuclear magnetic resonance
¹H-NMR Proton nuclear magnetic resonance
Ac Acetyl
Acac Acetylacetone
AcOH Acetic acid
ALK Anaplastic Lymphoma Kinase
Amphos Bis(di-tert-butyl(4-dimethylaminophenyl)phosphine)
B(cat) Catecholborane
B(Sia)₂ Bis(1,2-dimethylpropyl)borane
BBN Borabicyclo[3.3.1]nonane
BINAP (1,1′-Binaphthalene-2,2′-diyl)bis(diphenylphosphine)
Boc Tert-butyloxycarbonyl
BOM Benzyloxymethyl
br broad
br m Broad multiplet
br s Broad singlet
Bu Butyl
CaCO₃ Calcium carbonate
cat. Catalytic amount
Cbz Benzyloxycarbonyl
CDCl₃ Deuterated chloroform
CH₃CO₃H Peracetic acid
cod Cyclo-1,5-octadiene
cy Cyclohexyl
d Doublet
dba dibenzylideneacetone
DBU 1,8-Diazabicycloundec-7-ene
dcpp 1,3-Bis(dicyclohexylphosphanyl)propane
DIPEA N,N-Diisopropylethylamine or Hünig's base
DMA dimethylacetamide
DME dimethoxyethane
DMF Dimethylformamide
DMSO dimethylsulfoxide
DMSO-d₆ Dimethylsulfoxide deuterated
DPE-phos Bis(2-diphenylphosphinophenyl)ether
DPE-phos Bis(2-diphenylphosphinophenyl)ether
Dppb 1,4-Bis(diphenylphosphino)butane
Dppe 1,2-Bis(diphenylphosphino)ethane
Dppf 1,1′-bis(diphenylphosphanyl)ferrocene
Dppp 1,3-Bis(diphenylphosphanyl)propane
Eq. equivalent
Et Ethyl
Fmoc 9-Fluorenylmethyloxycarbonyl
g Gram(s)
h Hour(s)
H₂O₂ Hydrogen peroxide
HF Hydrogen fluoride
Hz Hertz
J Coupling constant
K₂CO₃ Potassium carbonate
KH Potassium hydride
KOAc Potassium acetate
LCMS liquid chromatography-mass spectrometry
m multiplet
M molarity/molar
mCPBA 3-chloroperoxybenzoic acid
Me Methyl
mg Milligram(s)
MIDA N-Methyliminodiacetic acid
min Minutes
ml milliliter
mol Mole(s)
Ms Mesyl
Na₂WO₄ Sodium tungstate dihydrate
NaH Sodium hydride
NBP 1-butyl-2-pyrrolidone
NMP N-methyl-2-pyrrolidone
Pd/Al Palladium on aluminum
Pd/C Palladium on Carbon
Pd-PEPPSI-Br [1,3-Bis(2,6-diisopropylphenyl)imidazole-2-ydilene](3-chloropyridyl)paladium(II) dichloride
PEPPSI Pyridine enhanced precatalyst preparation stabilization and initiation
Ph Phenyl
phos Phosphine
piv Pivaloyl
POM pivaloyloxymethyl
ppm parts per million
Pt/C Platinium on carbon
Rh/C Rhodium on carbon
s singlet
SEM Trimethylsilylethoxymethyl
Sept. septuplet
T° C. Temperature in celsius
TBHP tertbutylhydroperoxide
TBME Methyl tertbutylether
tBu tertbutyl
TES Triethylsilyl
Tf Triflate
THF tetrahydrofuran
TPAP tetrapropylammonium perruthenate
troc Trichloroethoxycarbonyl
Ts Tosyl
w/w Percentage by weight

### Examples

The Following examples are merely illustrative of the present disclosure and they should not be considered as limiting the scope of the disclosure in any way, as these examples and other equivalents thereof will become apparent to those skilled in the art in the light of the present disclosure, and the accompanying claims.

### Synthesis of 2-isopropoxy-5-methyl-4-(piperidin-4-yl)aniline dihydrochloride (C2, di-HCl salt) according to the following sequence

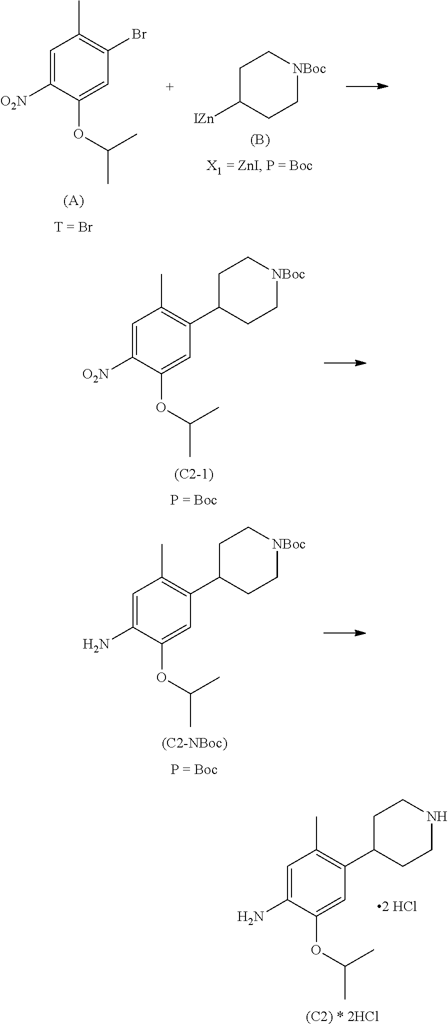

### Tert-butyl 4-(5-isopropoxy-2-methyl-4-nitrophenyl)piperidine-1-carboxylate (C2-1, P=Boc)

To a mixture of Pd(PPh₃)₂Cl₂ (69 mg, 0.099 mmol, 1.5 mol %), CuI (75 mg, 0.39 mmol, 6 mol %), 1-bromo-5-isopropoxy-2-methyl-4-nitrobenzene (1.80 g, 6.57 mmol) in tetrahydrofuran (19 ml), a solution of (1-(tert-butoxycarbonyl)piperidin-4-yl)zinc(II) iodide in tetrahydrofuran (4.95 g of a 0.9 M solution, 13.1 mmol, 2.0 eq.; prepared according to literature procedure) was added at 50° C. and the reaction mixture stirred for 21 hours at this temperature. After cooling to room temperature, saturated aqueous solution of NH₄Cl (50 ml) was added. The aqueous phase was extracted with ethyl acetate (3×70 ml), the combined organic phases washed with brine (80 ml) and dried over Na₂SO₄. The volatiles were removed under vacuum and the crude material was purified by silica gel chromatography (ethyl acetate/heptane) to yield tert-butyl 4-(5-isopropoxy-2-methyl-4-nitrophenyl)piperidine-1-carboxylate (1.96 g, 79% yield) as a brownish oil. ¹H-NMR (400 MHz, CDCl₃): δ=1.30 (d, J=6.1 Hz, 6H), 1.42 (s, 9H), 1.47-1.54 (m, 2H), 1.67-1.70 (m, 2H), 2.24 (s, 3H), 2.72-2.82 (m, 3H), 4.05 (br. m, 2H), 4.53 (sept, J=6.1 Hz, 1H), 6.79 (s, 1H), 7.55 (m, 1H) ppm.

### Tert-butyl 4-(4-amino-5-isopropoxy-2-methylphenyl)piperidine-1-carboxylate (C2-NBoc, P=Boc)

To a solution of tert-butyl 4-(5-isopropoxy-2-methyl-4-nitrophenyl)piperidine-1-carboxylate (1.96 g, 5.18 mmol) in ethanol (180 ml), Pd/C (10%, 0.4 g) was added and the reaction mixture was stirred for 5 hours at room temperature under a hydrogen atmosphere (4 bar). The hydrogen atmosphere was released, the reaction vessel purged with argon, and the mixture filtered through Celite®. The volatiles were removed under vacuum to obtain tert-butyl 4-(4-amino-5-isopropoxy-2-methylphenyl)piperidine-1-carboxylate (1.67 g, 92% yield) as a yellow oil, which was used in the next step without further purification.

### 2-Isopropoxy-5-methyl-4-(piperidin-4-yl)aniline dihydrochloride (C2, di-HCl salt)

Tert-butyl 4-(4-amino-5-isopropoxy-2-methylphenyl)piperidine-1-carboxylate (1.67 g) was dissolved in dichloromethane (10 ml) and the resulting solution treated with trifluoroacetic acid (2 ml). After 16 hours at room temperature, water (50 ml) was added. The phases were separated and the aqueous phase washed with dichloromethane (30 ml). The aqueous phase was neutralized with aqueous NaOH (1 M solution) and extracted with toluene (3×50 ml). To the combined toluene phases, HCl (3.8 ml of a 5 M solution in isopropanol) was added. After evaporation, 2-Isopropoxy-5-methyl-4-(piperidin-4-yl)aniline dihydrochloride was obtained (1.35 g, 88% yield). ¹H-NMR (400 MHz, D₂O): δ=1.24 (d, J=6.1 Hz, 6H), 1.76-1.96 (m, 4H), 2.22 (s, 3H), 3.04-3.14 (m, 3H), 3.45-3.49 (m, 2H), 4.71 (sept, J=6.1 Hz, 1H; overlapped with the solvent signal), 6.95 (s, 1H), 7.13 (s, 1H) ppm. ¹³C-NMR (100 MHz, D₂O): δ=17.4, 20.9, 28.6, 35.4, 44.4, 72.3, 112.2, 117.6, 125.4, 128.9, 144.6, 148.8 ppm.

### Synthesis of 2-isopropoxy-5-methyl-4-(piperidin-4-yl)aniline dihydrochloride (C2, di-HCl salt) by reduction of 4-(5-isopropoxy-2-methyl-4-nitrophenyl)pyridine (C)

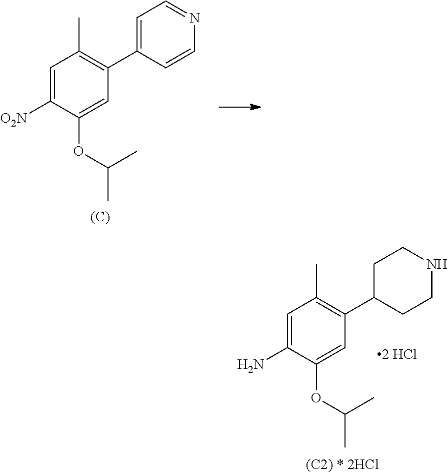

A reactor was charged with 4-(5-isopropoxy-2-methyl-4-nitrophenyl)pyridine (110 kg, 404 mol), 5 mol % Pt/C (33 kg, 50-70% H₂O) and acetic acid (2200 kg). The suspension was pressurized with hydrogen to 2 bar while maintaining the temperature below 30° C. After 2 hours the hydrogen pressure was increased to 6 bar and the mixture heated to 30° C. After full conversion, the hydrogen was released at room temperature and the reactor purged with nitrogen. The catalyst was filtered and rinsed with acetic acid (800 kg). The filtrate was partially concentrated, then toluene (2355 kg) was added, and distillation was continued; this step was repeated two more times (solvent switch to toluene). Additional toluene (312 kg) and isopropanol (359 kg) were added to the solution. HCl (154 kg of a 22% w/w solution in isopropanol) was dosed while maintaining the temperature below 40° C. After addition, the suspension was cooled to 25° C., held for at least 90 min, cooled to 0° C. and stirred for at least 90 min. The product was filtered, rinsed twice with toluene/isopropanol solution (2×240 kg, 20% w/w) and dried under vacuum to afford the product 2-isopropoxy-5-methyl-4-(piperidin-4-yl)aniline dihydrochloride (105 kg, 81% yield). ¹H NMR (400 MHz, DMSO-d6): δ=1.31 (d, J=4 Hz, 6H), 1.79 (m, 2H), 1.98 (qd, J=12, 4 Hz, 2H), 2.25 (s, 3H), 2.95-3.08 (br. m, 3H), 3.34 (br s, 1H), 4.66 (sept, J=4 Hz, 1H), 6.92 (s, 1H), 7.18 (s, 1H), 9.19 (br. s, 2H), 9.86 (br. s, 3H) ppm. ¹³C NMR (125 MHz, DMSO-d6): δ=18.1, 21.7, 28.4, 34.9, 43.5, 71.1, 112.1, 119.2, 125.6, 127.3, 144.0, 148.8 ppm.

### Synthesis of 4-(5-isopropoxy-2-methyl-4-nitrophenyl)pyridine (C)

a) One-Pot Borylation/Suzuki-Reaction

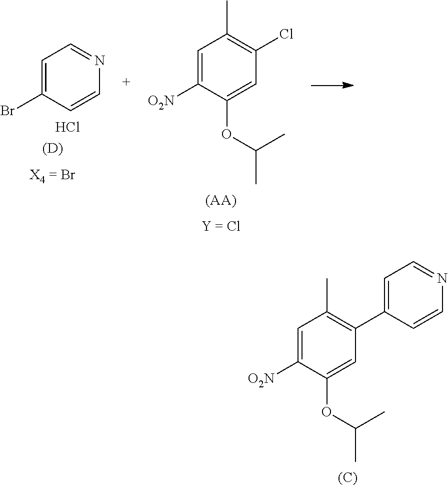

A mixture of 4-bromopyridine hydrochloride (1.0 g, 5.1 mmol, 1.2 eq.), bis(pinacolato)diboron (1.31 g, 5.1 mmol, 1.2 eq.), potassium acetate (1.68 g, 17.1 mmol, 4.0 eq.), 2-dicyclohexylphosphino-2′,4′,6′-triisopropylbiphenyl (=XPhos; 0.12 g, 6 mol %), chloro(2-dicyclohexylphosphino-2′,4′,6′-triisopropyl-1,1′-biphenyl)[2-(2′-amino-1,1′-biphenyl)]palladium(II) (=XPhos Precatalyst, 2ⁿd generation; 0.10 g, 3 mol %) and 1,4-dioxane (5 ml) was heated at 100° C. for 3 hours. Then aqueous K₂CO₃ (3.6 ml of a 3.6 M solution, 12.9 mmol, 3.0 eq.) and 1-chloro-5-isopropoxy-2-methyl-4-nitrobenzene (0.98 g, 4.3 mmol) was added and the reaction mixture further heated at 100° C. for 17 hours. After cooling to room temperature, the mixture was filtered over HyFlo® and washed with ethyl acetate (3×20 ml). The organic phase was washed with water (3×20 ml) and aqueous NaCl (20 ml of a 24% w/w solution) and dried over Na₂SO₄. The volatiles were removed under vacuum and the crude material was purified by silica gel chromatography (ethyl acetate/heptane) to yield 4-(5-isopropoxy-2-methyl-4-nitrophenyl)pyridine (0.95 g, 81% yield) as a yellow solid.

b) Suzuki-Coupling

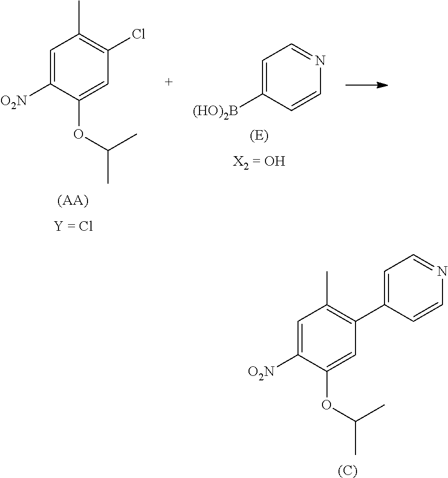

A mixture of K₂CO₃ (50.1 kg, 358 mol, 2.5 eq.), H₂O (93 kg), 1-chloro-5-isopropoxy-2-methyl-4-nitrobenzene (33.3 kg, 145 mol), pyridin-4-ylboronic acid (23.2 kg, 189 mol, 1.3 eq.), trans-dichlorobis(triphenylphosphine)palladium(II) (5.11 kg, 7.28 mol, 5 mol %) and 2-butanol (216 kg) was heated under an inert atmosphere at reflux for 3.5 hours. After cooling to 55° C., the mixture was filtered and the phases were separated. The organic phase was partially concentrated under vacuum, cooled to 50° C., and water (167 kg) was added. After further cooling to 5° C., the solids were filtered off. The crude product was purified by recrystallization from ethanol/water to yield 4-(5-isopropoxy-2-methyl-4-nitrophenyl)pyridine (52.9 kg, 71% yield) as a yellow solid. ¹H-NMR (400 MHz, CDCl₃): δ=1.40 (d, J=6.0 Hz, 6H), 2.23 (s, 3H), 4.65 (sept, J=6.1 Hz, 1H), 6.92 (s, 1H), 7.25-7.26 (m, 2H), 7.72 (s, 1H) 8.71-8.73 (m, 2H) ppm; ¹³C-NMR (100 MHz, CDCl₃): δ=19.2, 21.9, 73.1, 117.3, 123.6, 127.2, 127.4, 140.4, 144.4, 147.9, 149.2, 150.0 ppm.

### Synthesis of 2,5-dichloro-N-(2-(isopropylsulfonyl)phenyl)pyrimidin-4-amine (C3, X=Cl), according to the following sequence

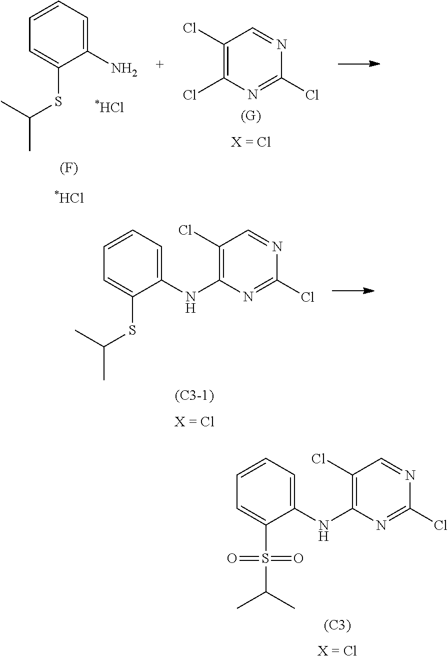

### 2,5-Dichloro-N-(2-(isopropylthio)phenyl)pyrimidin-4-amine (C3-1, X=Cl)

N,N-Diisopropylethylamine (31.0 g, 0.24 mol, 2.3 eq.) was added to a mixture of 2-(isopropylthio)aniline hydrochloride (21.3 g, 0.10 mol), 2,4,5-trichloropyrimidine (19.0 g, 0.10 mol, 1.0 eq.), in toluene (166 g) and n-butanol (17 g). The mixture was refluxed for about 22 hours. After cooling the reaction mixture to 25° C., water (70 g) was added, the phases were separated, and the organic phase was washed with water (70 g). The organic phase was concentrated under vacuum, followed by addition of ethanol (34 g). The mixture was refluxed and slowly cooled to 0-5° C., filtered and dried under vacuum to yield 2,5-dichloro-N-(2-(isopropylthio)phenyl)pyrimidin-4-amine (26.4 g, 81% yield). ¹H-NMR (400 MHz, CDCl₃): δ=1.30 (d, J=7.0 Hz, 6H), 3.18 (sept, J=6.7 Hz, 1H), 7.11-7.14 (m, 1H), 7.46-7.50 (m, 1H), 7.60-7.62 (m, 1H), 8.25 (s, 1H), 8.67-8.68 (m, 1H), 9.30 (br. s, 1H) ppm. ¹³C-NMR (100 MHz, CDCl₃): δ=23.3, 40.7, 114.9, 119.8, 122.9, 124.0, 130.4, 137.1, 139.8, 154.6, 156.0, 158.1 ppm.

### 2,5-Dichloro-N-(2-(isopropylsulfonyl)phenyl)pyrimidin-4-amine (C3)

To a solution of 2,5-dichloro-N-(2-(isopropylthio)phenyl)pyrimidin-4-amine (20.0 g, 63.7 mol) in ethyl acetate (180 g), peracetic acid (33.4 g of a solution in acetic acid; 5.7 mmol/g) was added while maintaining an internal temperature of 20-30° C. After a reaction time of 16 hours, ethyl acetate (90 g) was added, followed by aqueous sodium sulfite (112 g of a 11% w/w solution) and maintaining an internal temperature of below 45° C. The phases were separated and to the organic phase was added Water (63 g), followed by aqueous NaOH (25% w/w solution) to adjust the pH 7-8. The phases were separated, the organic phase was dried over MgSO₄ and concentrated under vacuum. Ethanol (217 g) was added the mixture refluxed. After slow cooling to 0-5° C., the precipitate was filtrated and dried under vacuum to yield 2,5-dichloro-N-(2-(isopropylsulfonyl)phenyl)pyrimidin-4-amine (19.5 g, 88% yield) as a white powder. ¹H-NMR (400 MHz, CDCl₃): δ=1.33 (d, J=6.7 Hz, 6H), 3.22 (sept, J=6.8 Hz, 1H), 7.31-7.35 (m, 1H), 7.72-7.76 (m, 1H), 7.92-7.94 (m, 1H), 8.31 (s, 1H), 8.63-8.65 (m, 1H), 10.07 (br s, 1H) ppm. ¹³C-NMR (100 MHz, CDCl₃): δ=153, 56.1, 115.2, 122.7, 124.2, 124.5, 131.5, 135.20, 137.4, 155.6, 156.3, 157.8 ppm.

### Synthesis of 2,5-dichloro-N-(2-(isopropylsulfonyl)phenyl)pyrimidin-4-amine (C3, X=Cl), according to the following sequence

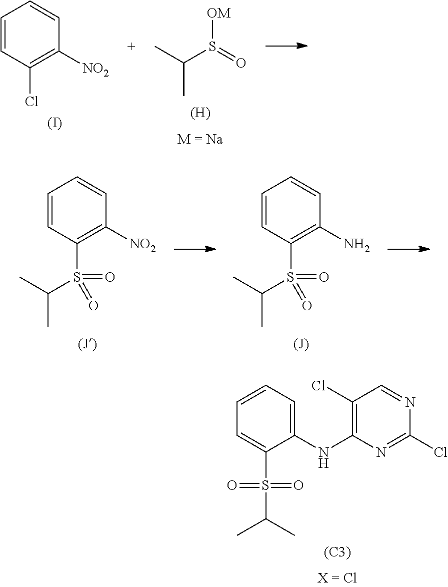

### 1-(Isopropylsulfonyl)-2-nitrobenzene (J′)

Sodium propane-2-sulfinate (2.48 kg, 19.1 mol, 1.5 eq.) and 1-chloro-2-nitrobenzene (2.00 kg, 12.7 mol) were dissolved in DMSO (5.5 kg) and heated to 85° C. for 12 hours. After cooling to 20° C., ice water (15 kg) was added followed by seeding with 1-(isopropylsulfonyl)-2-nitrobenzene (1 g). The reaction mixture was stirred for 30 min at 0-10° C., filtered and dried under vacuum to yield 1-(isopropylsulfonyl)-2-nitrobenzene (2.8 kg, 96% yield) as a grey solid.

### 2-(Isopropylsulfonyl)aniline (J)

A mixture of 1-(isopropylsulfonyl)-2-nitrobenzene (2.50 kg, 10.9 mol), ethanol (7.9 kg) and Pd/C (125 g, 5% w/w) in an autoclave was set under a hydrogen pressure of 3.4 bar and stirred for 48 hours at 40° C. After filtration of the catalyst, the filtrate was concentrated under vacuum to approximately 2.5 liters and the precipitate was filtered off. The filtrate was again concentrated under vacuum to approximately 0.8 liters and the precipitate was filtered off. The two filtered solids were combined and dried under vacuum to yield 2-(Isopropylsulfonyl)aniline (1.83 kg, 84% yield).

### 2,5-Dichloro-N-(2-(isopropylsulfonyl)phenyl)pyrimidin-4-amine (C3, X=Cl)

2,4,5-trichloropyrimidine (6.1 kg) was added onto 2-(isopropylsulfonyl)aniline (950 g, 4.77 mol) and DBU (181 g, 1.19 mol, 0.25 eq.), and the mixture was stirred for 7.5 hours at 80° C. After cooling to 25° C. n-heptane (1.9 kg) was added and the mixture was stirred for 30 min. The mixture was cooled to −5° C., filtered and washed with n-heptane (325 g). The crude product was suspended in ethanol (4.5 kg), stirred for 12 hours at 25° C. and then cooled to 0° C. After filtration the crude was dried under vacuum to yield 2,5-dichloro-N-(2-(isopropylsulfonyl)phenyl)pyrimidin-4-amine (1.07 kg, 65% yield). ¹H-NMR (400 MHz, CDCl₃): δ=1.33 (d, J=6.7 Hz, 6H), 3.22 (sept, J=6.8 Hz, 1H), 7.31-7.35 (m, 1H), 7.72-7.76 (m, 1H), 7.92-7.94 (m, 1H), 8.31 (s, 1H), 8.63-8.65 (m, 1H), 10.07 (br. s, 1H) ppm. ¹³C-NMR (100 MHz, CDCl₃): δ=153, 56.1, 115.2, 122.7, 124.2, 124.5, 131.5, 135.20, 137.4, 155.6, 156.3, 157.8 ppm.

### Synthesis of 2-isopropoxy-5-methyl-4-(piperidin-4-yl)aniline dihydrochloride (C2, di-HCl salt) by the following sequence

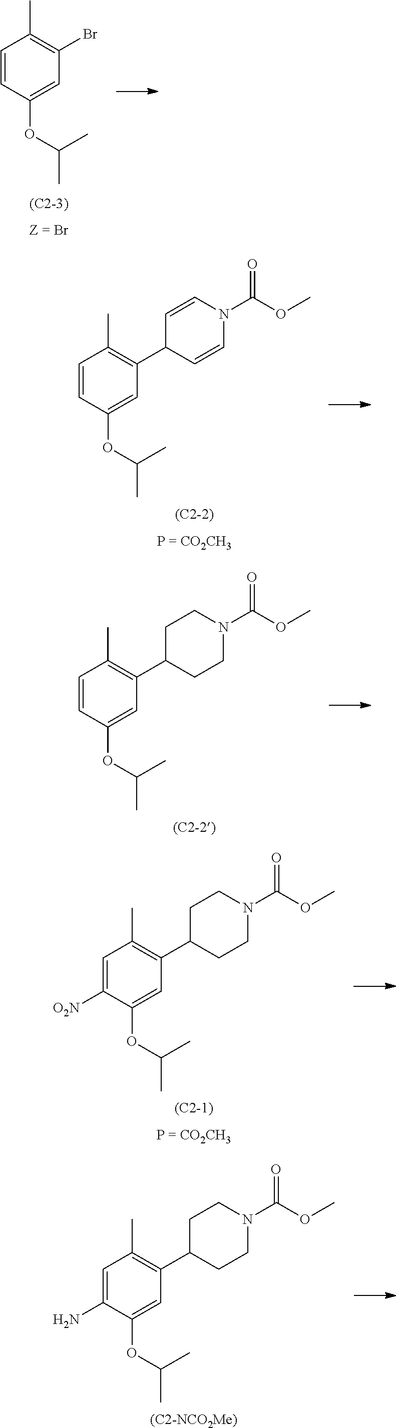

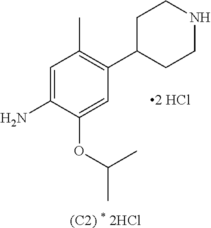

### Methyl 4-(5-isopropoxy-2-methylphenyl)pyridine-1(4H)-carboxylate (C2-2, P=CO₂CH₃)

To pre-dried LiCl (0.41 g, 9.6 mmol, 1.1 eq.), magnesium (0.28 g, 11.3 mmol, 1.3 eq.) and THF (13 ml) were added, followed by 2-bromo-4-isopropoxy-1-methylbenzene (2.00 g, 8.73 mmol). After 10 min at room temperature, the mixture was heated to 70° C. for 1 hour. The mixture was cooled to room temperature, stirred for 1 hour, and further cooled to −30° C. 1-(Methoxycarbonyl)pyridin-1-ium chloride (13.1 mmol; prepared from pyridine, acetyl chloride and copper(I)iodide according to literature procedure) was added, the resulting orange suspension was warmed to room temperature and stirred for 16 hours. TBME was added (80 ml), the organic phase washed with saturated aqueous NH₄Cl (3×30 ml), brine (30 ml) and was dried over Na₂SO₄. After removing the volatiles under vacuum, the obtained crude methyl 4-(5-isopropoxy-2-methylphenyl)pyridine-1(4H)-carboxylate (2.04 g, 86% yield) was used in the next step without further purification. ¹H-NMR (400 MHz, CDCl₃): δ=1.31 (d, J=6.1 Hz, 6H), 2.24 (s, 3H), 2.28 (s, 3H), 4.37 (m, 1H), 4.50 (sept, J=6.0 Hz, 1H), 4.93-4.96 (m, 1H), 5.03-5.07 (m, 1H), 6.62-6.68 (m, 2H), 6.82 (m, 1H), 7.04-7.06 (m, 1H), 7.28-7.31 (m, 1H) ppm.

### Methyl 4-(5-isopropoxy-2-methylphenyl)piperidine-1-carboxylate (C2-2′)

To a solution of 4-(5-isopropoxy-2-methylphenyl)pyridine-1(4H)-carboxylate (2.0 g, 7.5 mmol) in THF (20 ml) and methanol (50 ml), ammonium formate (10 g, 159 mmol) was added, followed by Pd/C (2.0 g, 10%). After stirring for 18 hours at room temperature, the mixture was filtered through Celite®, the filter cake washed with ethyl acetate (40 ml) and the filtrate charged with water (80 ml). The aqueous phase was extracted with ethyl acetate (3×50 ml) and the combined organic extracts dried over Na₂SO₄. The volatiles were removed under vacuum and the crude material was purified by silica gel chromatography (ethyl acetate/heptane) to yield methyl 4-(5-isopropoxy-2-methylphenyl)piperidine-1-carboxylate (2.1 g, 72% yield) as a yellow oil. ¹H-NMR (400 MHz, CDCl₃): δ=1.31 (d, J=6.1 Hz, 6H), 1.53-1.65 (m, 1H), 1.78-1.85 (m, 1H), 2.14 (s, 3H), 2.27 (s, 3H), 2.62 (m, 1H), 2.89 (m, 1H), 3.17 (m, 1H), 3.92-3.96 (m, 1H), 4.49 (sept, J=6.1 Hz, 1H), 4.78-4.82 (m, 1H), 6.64-6.69 (m, 2H), 7.04-7.06 (m, 1H) ppm.

### Methyl 4-(5-isopropoxy-2-methyl-4-nitrophenyl)piperidine-1-carboxylate (C2-1, P=CO₂CH₃)

A mixture of methyl 4-(5-isopropoxy-2-methylphenyl)piperidine-1-carboxylate (4.15 g) in acetic anhydride (51 ml) was cooled to −10° C. Nitric acid (1 ml, 65%) was added and the mixture stirred for 1 hour at −10° C. Another portion of nitric acid (1 ml, 65%) was added, stirred for 3 hours, and the mixture warmed to room temperature. After adding water (100 ml) to the mixture, the aqueous phase was extracted with ethyl acetate (3×80 ml). The combined organic phases were washed with saturated aqueous NH₄Cl (2×50 ml), brine (50 ml) and dried over Na₂SO₄. The volatiles were removed under vacuum and the crude material purified by silica gel chromatography (ethyl acetate/heptane) to yield methyl 4-(5-isopropoxy-2-methyl-4-nitrophenyl)piperidine-1-carboxylate (2.1 g, 72% yield) as a yellow oil. LCMS: m/z (M+1) 337.2 [M+H]⁺.

### Methyl 4-(4-amino-5-isopropoxy-2-methylphenyl)piperidine-1-carboxylate (C2-NCO₂Me)

Pd/C (0.5 g, 10%) was added to a solution of 4-(5-isopropoxy-2-methyl-4-nitrophenyl)piperidine-1-carboxylate (3.68 g, 10.9 mmol) in ethanol (200 ml). The mixture was stirred for 5 hours under a hydrogen atmosphere at 5 bar. The reaction mixture was purged with nitrogen, filtered through Celite®, and washed with ethyl acetate (50 ml). The filtrate was extracted with aqueous HCl (2×30 ml, 1 M solution). The aqueous phases were neutralized with aqueous NaOH (1 M solution) and extracted with ethyl acetate (3×50 ml). The volatiles were removed under vacuum and the crude material was purified by silica gel chromatography (ethyl acetate/heptane) to yield methyl 4-(4-amino-5-isopropoxy-2-methylphenyl)piperidine-1-carboxylate (3.36 g, 51% yield). ¹H-NMR (400 MHz, CDCl₃): δ=1.25 (d, J=6.1 Hz, 6H), 1.47 (br. m, 2H), 1.65-1.68 (m, 2H), 2.13 (s, 3H), 2.65-2.81 (m, 3H), 3.59 (br. s, 2H), overlapped by 3.65 (s, 3H), 4.22 (br. s, 2H), 4.37 (sept, J=6.1 Hz, 1H), 6.46 (s, 1H), 6.53 (s, 1H) ppm.

### 2-Isopropoxy-5-methyl-4-(piperidin-4-yl)aniline dihydrochloride (C2, di-HCl salt)

Aqueous HCl (26 ml of a 8 M solution) was added to methyl 4-(4-amino-5-isopropoxy-2-methylphenyl)piperidine-1-carboxylate (1.00 g, 3.26 mmol) and the mixture was stirred for 16 hours at room temperature. After washing with dichloromethane (30 ml), the aqueous phase was neutralized with aqueous NaOH (1 M solution). The neutralized aqueous phase was extracted with toluene (3×50 ml) and the combined organic extracts were treated with HCl (2.6 ml of a 5 M solution in isopropanol). Evaporation of the volatiles under vacuum yielded 2-isopropoxy-5-methyl-4-(piperidin-4-yl)aniline dihydrochloride (1.05 g, 49% yield). LCMS: m/z (M−2HCl) 337.2 [M+H]; ¹H-NMR (400 MHz, D₂O): 6=1.24 (d, J=6.1 Hz, 6H), 1.76-1.96 (m, 4H), 2.22 (s, 3H), 3.04-3.14 (m, 3H), 3.46-3.49 (m, 2H), 4.71 (sept, J=6.1 Hz, 1H; overlapped with the solvent signal), 6.95 (s, 1H), 7.13 (s, 1H) ppm.

### Synthesis of 5-chloro-N2-(2-isopropoxy-5-methyl-4-(piperidin-4-yl)phenyl)-N4-(2-(isopropylsulfonyl) phenyl)pyrimidine-2,4-diamine (ceritinib)

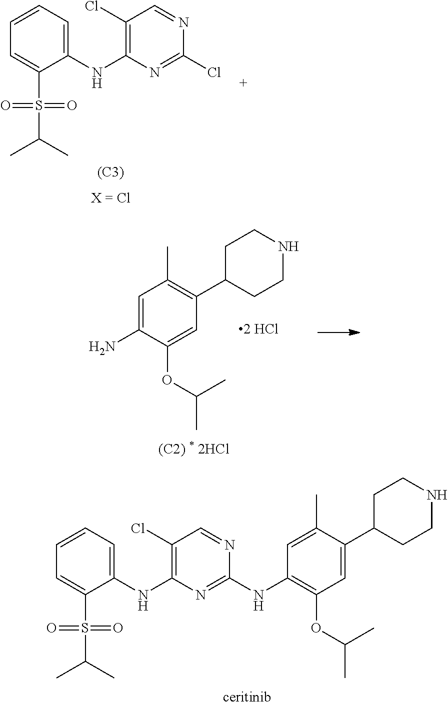

Isopropanol (445 kg) was added to 2,5-dichloro-N-(2-(isopropylsulfonyl)phenyl)pyrimidin-4-amine (70.2 kg, 203 mol, 1.15 eq.) and 2-Isopropoxy-5-methyl-4-(piperidin-4-yl)aniline dihydrochloride (56.7 kg, 176 mol). The mixture was heated for approx. 16 hours at reflux. Water (47 kg) was added and the mixture cooled to 0° C. The solid was filtered and washed with isopropanol/water. To the wet product, isopropanol (680 kg) and water (55 kg) was added and the slurry heated to reflux. The obtained clear solution was cooled to 0° C., filtered and dried under vacuum to yield 5-chloro-N2-(2-isopropoxy-5-methyl-4-(piperidin-4-yl)phenyl)-N4-(2-(isopropylsulfonyl)phenyl)pyrimidine-2,4-diamine dihydrochloride (=ceritinib dihydrochloride; 84.5 kg [t.q., contains 10% w/w isopropanol], 76.0 kg [100%], 68% yield).

Ethanol (155 kg) and water (113 kg) were added to 5-chloro-N2-(2-isopropoxy-5-methyl-4-(piperidin-4-yl)phenyl)-N4-(2-(isopropylsulfonyl)phenyl)pyrimidine-2,4-dihydrochloride; 45.0 kg [t.q., contains 10% w/w isopropanol], 40.5 kg [100%], 64.2 mol) and the mixture heated to 55° C. Aqueous NaOH (147 L of a 1 M solution, 2.3 eq.) was added slowly and then cooled to 20° C. After filtration, the product was recrystallized from ethanol and dried under vacuum to yield ceritinib (33.2 kg, 93% yield based on ceritinib dihydrochloride) as an almost white powder. ¹H-NMR (400 MHz, CDCl₃): δ=1.33 (d, J=6.8 Hz, 6H), 1.38 (d, J=6.1 Hz, 6H), 1.59-1-78 (m, 5H), 2.18 (s, 3H), 2.75-2.83 (m, 3H), 3.20-3.24 (m, 2H), overlaps 3.28 (sept, J=6.8 Hz, 1H), 4.56 (sept, J=6.1 Hz, 1H), 6.82 (s, 1H), 7.25-7.29 (m, 1H), 7.56 (br. s, 1H), 7.64 (m, 1H), 7.94 (m, 1H), 8.01 (br. s, 1H), 8.16 (br. s, 1H), 8.60 (m, 1H), 9.51 (br. s, 1H) ppm. ¹³C- NMR (100 MHz, CDCl₃): δ=15.4, 18.9, 22.3, 33.9, 38.6, 47.5, 55.4, 71.4, 105.7, 111.0, 120.6, 123.1, 123.7, 124.9, 126.7, 127.3, 131.2, 134.7, 138.3, 138.5, 144.7, 155.3, 155.4, 157.5 ppm, Elemental analysis: calculated (%) for C₂₈H₃₆ClN₅O₃S: C, 60.26, H, 6.50, N, 12.55, O, 8.60 Cl, 6.35, S, 5.74, found: C, 60.15, H, 6.45, N, 12.72, O, 8.58, Cl, 6.43, S, 5.67.)

1-61: (canceled)

62. A process for preparing a compound of formula (C2-1), 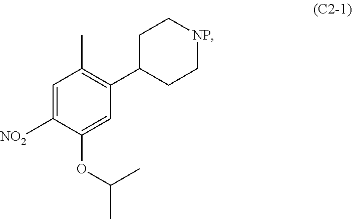
the process comprising the step of reacting a compound of formula (A) 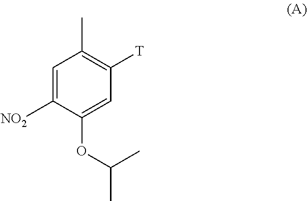
with a compound of formula (B) in a solvent 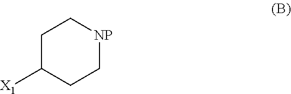
in the presence of at least one catalyst and optionally a co-catalyst or additive, wherein
P is a protecting group;
T and X1 are independently selected from the group consisting of Cl, Br, I, OTf, OTs, OPiv, Mg, Al, Zn, Zr, B, Sn, Si, ZnCl, ZnBr, ZnI, ZnAlkyl, B(OH)₂, B(OC(CH₃)₂C(CH₃)₂O), 9-BBN, B(Sia)₂, B(Cat), B(Cy)₂, BF₃⁻, B(MIDA);
the catalyst is Pd(PPh₃)₂Cl₂; and
the co-catalyst or additive is CuI.

63. The process for preparing a compound of formula (C2-1) according to claim 1, wherein T is Br and X1 is ZnI.

64. A process for preparing a compound of formula (C2-1), the process comprising the steps of reacting a compound of formula (C2-3) 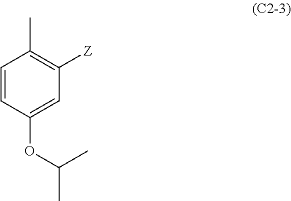
with 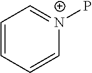
(pyridinium-P) in a solvent to produce a compound (C2-2); 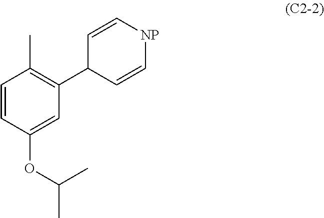
and transforming the compound (C2-2) to form the compound of formula (C2-1) 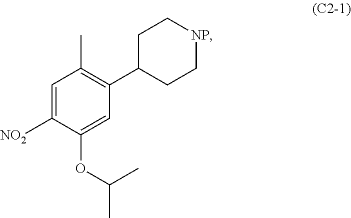
wherein
P is a protecting group and
Z is selected from MgBr, MgCl, MgI, Mg, ZnCl, ZnBr, ZnI or Zn(Alkyl).

65. A process for preparing a compound of formula (C2), or a salt thereof, comprising the step of reducing a compound of formula (C) in a solvent 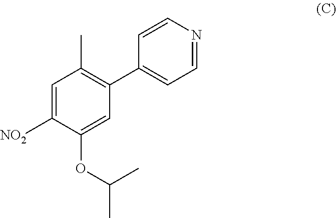 [SMILES: Cc1cc([N+](=O)[O-])c(OC(C)C)cc1-c1ccncc1]
in the presence of at least one catalyst and hydrogen, wherein the catalyst is Pt/C or Pd/Al.

66. The process for preparing a compound of formula (C2), or a salt thereof, according to claim 4, wherein the reaction is performed in acetic acid in the presence of Pd/Al and hydrogen at 60° C. and a pressure of 80 bar.

67. The process for preparing a compound of formula (C2), or a salt thereof, according to claim 4, wherein the reaction is performed in the presence of Pt/C and hydrogen at 10 to 50° C. and a pressure of 1 to 10 bar.

68. A one-pot process for preparing a compound of formula (C), or a salt thereof, comprising the step of reacting a compound of formula AA in a solvent 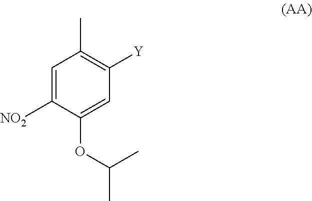
with a compound of formula D in the presence of X₃B(X₂)₂, at least one catalyst, a base, and 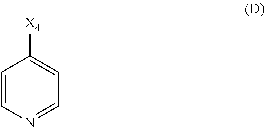
without isolating a compound of formula (E), 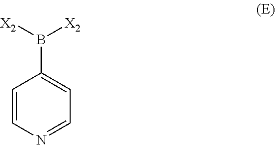
wherein
Y is selected from Cl, Br, I, OTf, OTs, OPiv and OMs;
X₄ is selected from Cl, Br, I, OTf, OTs, OPiv and OMs;
B(X₂)₂ is selected from B(OH)₂, B(OC(CH₃)₂C(CH₃)₂O), 9-BBN, B(Sia)₂, B(cat), B(Cy)₂; and X₃ is H or B(X₂)₂.

69. The process for preparing the compound of formula (C), or a salt thereof, according to claim 7, wherein Y is Cl or Br; B(X₂)₂ is B(OH)₂ or B(OC(CH₃)₂C(CH₃)₂O) and X₃ is B(X₂)₂.

70. A process for preparing a compound of formula (C3-1) 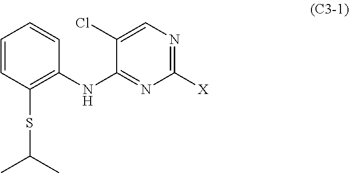
comprising the step of reacting a compound of formula (F) 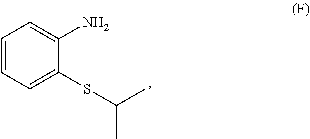
or a salt thereof,
with a compound of formula (G) 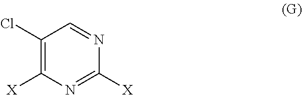
in the presence of a base, wherein
X is selected from the group consisting of halogen (Br, Cl, I), alkoxy, aryloxy, alkylthio, sulfinyl and sulfonyl; and
the base is selected from DIPEA or DBU.

71. A process for preparing a compound of formula (C3), comprising the process of claim 9 and oxidizing the obtained (C3-1) 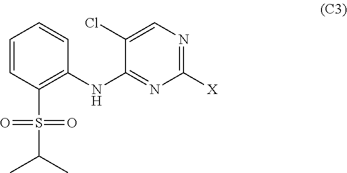
in a solvent, wherein X is selected from halogen (Br, Cl, I), alkoxy, aryloxy, alkylthio, sulfinyl and sulfonyl; and the oxidizing agent is selected from CH₃CO₃H or H₂O₂ and Na₂WO₄.

72. The process for preparing a compound of formula (C3) according to claim 10, wherein the process is carried out in ethyl acetate, in the presence of CH₃CO₃H as a solution in CH₃CO₂H, at the temperature between 20-40° C.

73. The process for preparing a compound of formula (C3) according to claim 10, wherein the process is carried out in methanol, in the presence of H₂O₂ and Na₂WO₄, at the temperature between 20 and 70° C.

74. A process for preparing a compound of formula (C3), the process comprising the steps of
(i) reacting a compound of formula (H) 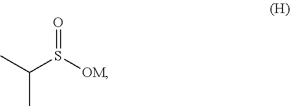
with a compound of formula (I) to form an intermediate; 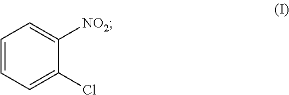
(ii) reducing the intermediate; and
(iii) reacting the reduced intermediate with a compound of formula 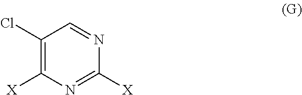
in the presence of a base, wherein X is selected from the group consisting of halogen (F, Cl, Br, I), alkoxy, aryloxy, alkylthio, sulfinyl and sulfonyl and M is selected from Li, Na, K, 0.5 Zn, 0.5 Ca; and
the base is DBU or DIPEA.

75. The process for preparing ceritinib, or a salt thereof, wherein the compound of formula (C2), or a salt thereof, is reacted with the compound of formula (C3) in a solvent, in the absence of a base, wherein the solvent is selected from 1,4-dioxane, tetrahydrofuran (THF), 2-methyl tetrahydrofuran, diethyl ether, toluene, N-methyl-2-pyrrolidone (NMP), 1-butyl-2-pyrrolidone (NBP), acetonitrile, acetone, dimethylcarbonate, ethylacetate, isopropylacetate, tertbutylacetate, water, methanol, ethanol, n-propanol, isopropanol, n-butanol, 2-butanol, tert-butanol and toluene, or mixtures thereof, and the optional base is selected from Na₂CO₃, K₂CO₃, Cs₂CO₃, NaHCO₃, KHCO₃, trimethylamine, DIPEA, DBU, Na₃PO₄ and K₃PO₄.

76. A compound of formula (C2-2) 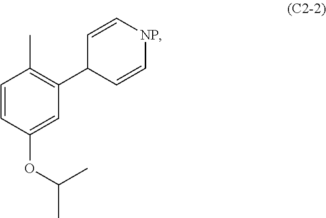
wherein
P is a protecting group.
